(19) United States
(12) Patent Application Publication
(10) Pub. No.: US 2023/0381244 A1
(43) Pub. Date: Nov. 30, 2023

(54) Methods and Systems for Improved Therapies Using Photo-Activated Stem Cells

(71) Applicant: Todd Frank Ovokaitys, Carlsbad, CA (US)

(72) Inventor: Todd Frank Ovokaitys, Carlsbad, CA (US)

(21) Appl. No.: 18/295,661

(22) Filed: Apr. 4, 2023

Related U.S. Application Data
(63) Continuation-in-part of application No. 17/643,279, filed on Dec. 8, 2021.
(60) Provisional application No. 63/362,410, filed on Apr. 4, 2022.
(60) Provisional application No. 63/180,742, filed on Apr. 28, 2021.
(60) Provisional application No. 63/122,836, filed on Dec. 8, 2020.
(60) Provisional application No. 63/122,831, filed on Dec. 8, 2020.

(51) Int. Cl. A61K 35/545; C12N 5/0735; C12N 13/00; A61P 9/04

(52) U.S. Cl. CPC A61K 35/545; C12N 5/0606; C12N 2500/32; A61P 9/04; C12N 13/00

(57) ABSTRACT

Platelet rich plasma containing human very small embryonic-like stem cells (hVSEL) is treated with amplitude-modulated pulses of laser light having a predefined wavelength for a predefined time period, where the predefined wavelength ranges from 300 nm to 1000 nm. Treatment of the platelet rich plasma using this method results in an unexpectedly high degree of proliferation of the hVSEL in the platelet rich plasma, resulting in reduction of biological age, and treatment of diseases, when administered to a patient.

### CROSS-REFERENCE

The present application relies on U.S. Patent Provisional Application No. 63/362,410, titled “Methods and Systems for Increased Production of Stem Cells”, and filed on Apr. 4, 2022, for priority.

In addition, the present application is a continuation-in-part of U.S. patent application No. 17/643,279, titled “Methods and Systems for Increased Production of Stem Cells”, and filed on Dec. 8, 2021, which, in turn, relies on the following U.S. Patent Provisional Applications, for priority, which are all herein incorporated by reference in their entirety:

U.S. Patent Provisional Application No. 63/122,831, titled “Methods and Systems for Increased Production of Stem Cells”, and filed on Dec. 8, 2020;

U.S. Patent Provisional Application No. 63/122,836, titled “Methods and Systems for Increased Production of Stem Cells”, and filed on Dec. 8, 2020; and

U.S. Patent Provisional Application No. 63/180,742, titled “Methods and Systems for Increased Production of Stem Cells”, and filed on Apr. 28, 2021.

In addition, the present application relates to United States Patent Publication Number 20210207121, (U.S. patent application Ser. No. 17/146,849), titled “Methods and Systems for Generation, Use, and Delivery of Activated Stem Cells”, filed on Jan. 12, 2021, which is a continuation of U.S. Pat. No. 10,907,144, titled “Methods and Systems for Generation, Use, and Delivery of Activated Stem Cells”, issued on Feb. 2, 2021, which, in turn, is a continuation of issued U.S. Pat. No. 10,202,598, of the same title, issued on Feb. 12, 2019, which, in turn, is a continuation-in-part of U.S. Pat. No. 9,999,785, titled “Method and System for Generation and Use of Activated Stem Cells” and issued on Jun. 19, 2018, which, in turn, relies on U.S. Patent Provisional Application No. 62/006,034, filed on May 30, 2014, for priority. The '598 patent further relates to the following United States Provisional Patent Applications, which are also herein incorporated by reference in their entirety: U.S. Provisional Patent Application No. 62/321,781, entitled “Method and System for Generation and Use of Activated Stem Cells”, and filed on Apr. 13, 2016; and, U.S. Provisional Patent Application No. 62/254,220, entitled “Method and System for Generation and Use of Activated Stem Cells”, and filed on Nov. 12, 2015.

The above-mentioned applications are herein incorporated by reference in their entirety.

### FIELD

The present specification discloses methods and systems for the improved production of stem cells and, in particular, the use of modulated laser impulses to increase the proliferation of stem cells to reverse the biological aging process and/or reduce biological age.

### BACKGROUND

Tissues that undergo rapid physiological turnover, or that are damaged by trauma or disease, need repair by a wide range of stem cells present in the human body. These stem cells include hemopoietic stem cells, mesenchymal stem cells and other more recently discovered stem cells such as neural stem cells in the Central Nervous System (CNS) and bronchio-alveolar stem cells in the lungs. In addition to these naturally occurring stem cells there are ‘man made’ stem cells such as human Embryonic Stem Cells (hESC) and Induced Pluripotent Stem Cells (iPSC). Despite these discoveries, only one stem cell type (the hemopoietic stem cell) is currently used in routine clinical practice in the treatment of hematological malignancy such as leukemia. The remainder of the stem cell types are still either at the basic research stage or, at best, in clinical trials.

A stem cell that is present in early embryonic development is referred to as a Primordial Germ Cell (PGC). PGCs arise before gastrulation in the proximal epiblast and find their way to the genital ridge (via extraembryonic tissue and the primitive streak) of the developing embryo while retaining broad, cross-germ-layer differentiation ability. Primordial germ cells (PGCs) are therefore the precursors of sperm and oocytes, specified around the time of gastrulation. PGCs are induced by signals from the surrounding embryonic tissues to the equipotent epiblast cells that give rise to all cell types. Gastrulation is a key phase in embryonic development when pluripotent stem cells differentiate into the three primordial germ layers namely: ectoderm, mesoderm and endoderm, wherein cells in each germ layer differentiate into tissues and embryonic organs. The ectoderm gives rise to the nervous system and the epidermis, among other tissues. The mesoderm gives rise to the muscle cells and connective tissue in a human body. The endoderm gives rise to the gut and a plurality of internal organs.

The migration of PGCs results in the development of the first hemangioblasts which are precursors to hemopoietic stem cells (HSC) and endothelial progenitor cells (EPC) in the yolk sac of the developing embryo. The next stage of development is that the PGCs migrate to the genital ridges of the embryo and on through the aorto-gonado-mesonephros (AGM) where the first definitive HSCs are found in the aortic endothelium. It has thus been suggested that human very small embryonic-like (hVSEL) stem cells are produced by migrating PGCs. This concept may further be supported because both PGCs and hVSEL stem cells have been shown to be capable of producing HSC and EPC. The current evidence appears to suggest that hVSEL stem cells may be precursors to HSC, and, if so, play a critical part in hemopoiesis from early embryonic development and throughout normal life. It has been established that hVSEL stem cells, since they are pluripotent, may be the precursors of other stem cells in the body. hVSEL stem cells also persist in peripheral blood throughout life. Hence, it is possible to obtain autologous hVSEL stem cells from any patient at any age.

VSEL stem cells were first identified in mouse bone marrow and are described as small (1-4 μm) non-hemopoietic cells with a high nuclear to cytoplasm ratio. They express similar surface antigens to pluripotent embryonic stem cells. hVSEL stem cells were first identified in umbilical cord blood and have been shown to be CXCR4+, CD34+, CD133+, Oct4+, SSEA4+and lin−, CD45−. hVSEL stem cells have subsequently been shown to be present in peripheral blood and bone marrow and in leukapheresis samples taken following granulocyte—colony stimulating factor (G-CSF) administration. hVSEL stem cells have since been described in the peripheral blood at a concentration of 800-1300 cells/mL.

hVSEL stem cells are a population of epiblast-derived cells created during embryonic gastrulation. hVSEL stem cells may be important in the long-term production of CD34+ hematopoietic stem cells in the bone marrow and may contribute to repair in experimental myocardial infarction (MI). hVSEL stem cells also persist in peripheral blood throughout life. Accordingly, it may be possible to obtain autologous hVSEL stem cells from any patient at any age, thereby enabling their use in regenerative medicine, simplifying procedures, saving money and reducing adverse reactions associated with allogeneic cells. hVSEL stem cells may also be a viable option to potentially developing pancreatic tissue and human gametes. With correct handling and administration, hVSEL stem cells could play a critical part in translational regenerative medicine in the future.

The use of laser light as a method of activation of stem cells, referred to as photo-biomodulation, has been reported recently. It has also been shown that photo-biomodulation may improve tissue regeneration and the proliferation, migration and differentiation of stem cells. For example, it has been demonstrated that 420 nm and 540 nm laser wavelengths stimulated osteogenic differentiation whereas the other wavelengths did not. Broadband visible light (low-level visible light) has been shown to increase proliferation of bone marrow mesenchymal (MSC) in vitro. The photobiomodulation effects of laser light on dental pulp MSC, human adipose MSC and epithelial colony forming units have also been described.

Flow cytometry is often used to assess laser treated biological samples for cell proliferation. Surface antigens Oct 3/4, SSEA4 and CXCR4 in the lineage negative (Lin−) compartment are assessed using flow cytometry. Of these three markers, it is known that CXCR4 may be blocked from binding by flow cytometry antibodies via its antagonistic ligand, the Endogenous Peptide Inhibitor EPI-X4. This blocking of CXCR4 disrupts or hinders accurate assessment using flow cytometry.

Additionally, organisms have a biological age, which is distinct and separate from the organism's chronological age. The biological age is determined at a cellular level, and may depend on several factors such as lifestyle, environment, and genetics, among other factors. Humans who have a younger biological age as compared to their chronological age are at a lower risk of experiencing age-related diseases. There are well known techniques for measuring biological age. In one example, telomere length is used as an indicator of biological age. In another example, DNA methylation is assessed, which involves a test to determine biological age by measuring intrinsic epigenetic age; thereby relating methylation status to biological age. It has been determined that DNA methylation age is close to zero for embryonic and induced pluripotent stem cells.

Platelet-rich plasma (PRP) is an important component of human blood as the platelets in PRP have a potential role in the anti-inflammatory and regenerative properties which have been observed when PRP is used clinically. Platelets are non-nucleated cells which are derived from the megakaryocyte located in the bone marrow and they contain four types of granules: i) alpha granules containing the adhesive proteins fibrinogen, vitronectin, thrombospondin and von Willebrand Factor (VWF). In addition, alpha granules contain growth factors and cytokines which mediate wound repair, inflammation, and angiogenesis; ii) dense (or delta) granules containing ADP, ATP, calcium, serotonin, polyphosphate and pyrophosphate; iii) lysosomes containing hexosaminidase, arylsulfatase, β-glucuronidase, β-galactosidase, acid phosphatase and cathepsins; iv) T (or tubular) granules containing TLR9, PDI and VAMP-846 which are thought to be an alpha granule subtype.

The plasma component of PRP is also important in the overall potential therapeutic action of PRP. The plasma in PRP contains high concentrations of growth factors and cytokines such as a wide range of interleukins, RANTES, PDGF, VEGF, GM-CSF, MIP 1b and CXCL chemokine (IP-10). These are wide-ranging cytokines and growth factors which when in concentrated form in PRP enable differentiation, proliferation, tissue morphogenesis and chemotaxis in tissue healing. Autologous PRP also has use in fertility treatment where the added component of possible endocrine action of PRP may be active. Therefore, PRP contains a complex and interactive range of cytokines and growth factors.

PRP also contains hVSEL stem cells. Research has shown the presence of CXCR4+, SSEA4+, Oct 3/4+, CD45−, Lin− hVSEL pluripotent stem cells in PRP derived from human peripheral blood. PRP contains hVSEL stem cells and that PRP is therefore a readily available source of functioning pluripotent stem cells. The standardization of the production of PRP is required to ensure homogeneity, safety and efficacy of PRP treatment.

The activation technologies described above may well also activate hVSEL stem cells. What is needed, therefore, is a method of increasing the amount of stem cells per volume of platelet rich plasma (PRP) fluid. Most autologous PRP treatments involve the collection of peripheral blood into a citrate dextrose anticoagulant, centrifugation at room temperature, followed by a simple reinfusion of the room temperature PRP back into the patient. Such preparation of PRP may result in premature platelet activation which can be modulated by introducing thrombin into the PRP. In some cases, pulsed electrical fields have been used to stimulate platelet activation and growth factor release in PRP. More recently, the voltage, pulse width, and calcium concentration has been shown to modulate the release of growth factors, serotonin and hemoglobin. It has also been reported that PRP may be activated by carrying out processing at 4° C. which promotes wound healing. The current evidence seems to indicate that PRP levels can be ‘improved’ by activation interventions of various types, the focus of which is to activate platelets which, in turn, increases the efficacy of PRP.

Specifically, what is needed is a method of using modulated laser photobiomodulation to increase the proliferation of peripheral blood hVSEL stem cells. What is also needed is a method to unblock CXCR4 thus making it readily available for binding to flow cytometry antibodies. Additionally, it is desirable to have compositions and methods for slowing down or reversing the biological clock so that the biological age of an organism is less than the chronological age of the same organism. Furthermore, what is needed are compositions and methods for the effective treatment of various diseases including cardiac disease. Finally, what is needed are compositions and methods for the treatment of in-born errors of metabolism and/or genetic encoding errors created by single nucleotide polymorphisms (SNP).

### SUMMARY

The following embodiments and aspects thereof are described and illustrated in conjunction with systems, tools and methods, which are meant to be exemplary and illustrative, not limiting in scope. The present specification discloses numerous embodiments.

The present specification is directed to a method of treatment of a disease of a patient, wherein said disease is characterized by at least one unique marker, comprising: determining the patient has said disease based at least in part on a value of said at least one unique marker; proliferating stem cells of the patient, wherein said proliferation comprises: obtaining platelet rich plasma from the patient, wherein the platelet rich plasma has a first quantity of stem cells; exposing the platelet rich plasma with the first quantity of stem cells to modulated pulses of laser light having a predefined wavelength and for a predefined period of time; and, after said exposure, harvesting from said platelet rich plasma a second quantity of stem cells, wherein the second quantity of stem cells is greater than the first quantity; administering a composition comprising, at least in part, said second quantity of stem cells; evaluating said at least one unique marker; determining whether said at least one unique marker is at an acceptable level; if said at least one unique marker is at an acceptable level, ending said method of treatment; and if said at least one unique marker is not at an acceptable level, waiting a period of time and repeating each of the determining, proliferating and administering steps.

Optionally, preparing the platelet rich plasma comprises: placing the patient's blood into a plurality of tubes; centrifuging the plurality of tubes for a predefined period of time to produce the platelet rich plasma; and aliquoting the produced platelet rich plasma into a sterile tube. Optionally, the method further comprises shaking the sterile tube after aliquoting.

Optionally, the method further comprises shaking the platelet rich plasma after treating with modulated pulses of laser light.

Optionally, the predefined wavelength ranges from 300 nm to 1000 nm.

Optionally, the predefined wavelength is in a range of 580 nm to 770 nm.

Optionally, the predefined period of time ranges from 1 minute to 5 minutes.

Optionally, the second quantity of stem cells is greater than the first quantity by 10% up to 400%.

Optionally, repeating each of the determining, proliferating and administering steps occurs 1 day to 6 months after said administering of the composition comprising, at least in part, said second quantity of stem cells.

Optionally, the marker is an ejection fraction percentage and said ejection fraction percentage increases by at least 8% after said administering of the composition comprising, at least in part, said second quantity of stem cells.

Optionally, the marker is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and said marker improves by at least 8% after said administering of the composition comprising, at least in part, said second quantity of stem cells.

Optionally, the disease comprises at least one of a cardiac disease, a neurodegenerative disease, a musculoskeletal trauma, a neurological trauma, type 2 diabetes, liver disease, lung disease, pancreatic disease, a psychosis disorder, or a psychiatric disorder.

The present specification also discloses a method of treating a disease in a patient characterized by a predefined parameter, the method comprising: characterizing the predefined parameter for determining said parameter to be out of a predefined acceptable parameter range; obtaining platelet rich plasma comprising a first quantity of autologous hVSELs from the patient; increasing a number of said hVSEL stem cells from the first quantity to a second quantity by applying to said platelet rich plasma pulses of modulated laser light; harvesting the second quantity of hVSEL stem cells; administering the second quantity of hVSEL stem cells to the patient; reevaluating the patient's predefined parameter; determining if the patient's predefined parameter is at an acceptable level; repeating at predefined intervals of time each of the obtaining, increasing, harvesting, and administering steps if the patient's predefined parameter is not at the acceptable level; and concluding said method of treating the patient if the patient's predefined parameter is at the acceptable level.

Optionally, after performing each of the obtaining, increasing, harvesting, and administering steps, the patient's predefined parameter improves by at least 8%.

Optionally, the second quantity of hVSEL stem cells is greater than the first quantity by 10% up to 400%.

Optionally, said predefined intervals of time are in a range of 1 day to 6 months, or any time increment therein.

Optionally, the pulses of modulated laser light have a predefined wavelength in a range of 580 nm to 770 nm.

Optionally, the predefined parameter is an ejection fraction percentage and said ejection fraction percentage increases by at least 8% after said administering the second quantity of hVSEL stem cells.

Optionally, the predefined parameter is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and said predefined parameter improves by at least 8% after said administering the second quantity of hVSEL stem cells.

Optionally, the disease comprises at least one of a cardiac disease, a neurodegenerative disease, a musculoskeletal trauma, a neurological trauma, type 2 diabetes, liver disease, lung disease, pancreatic disease, a psychosis disorder, or a psychiatric disorder.

The present specification also discloses a method of producing a composition that, when administered to a patient, treats a disease of a patient, wherein said disease is characterized by at least one unique marker, the method comprising: determining the patient has said disease based at least in part on a value of said at least one unique marker; proliferating stem cells of the patient, wherein said proliferation comprises: obtaining platelet rich plasma from the patient, wherein the platelet rich plasma has a first quantity of stem cells; exposing the platelet rich plasma with the first quantity of stem cells to modulated pulses of laser light having a predefined wavelength and for a predefined period of time; and, after said exposure, harvesting from said platelet rich plasma a second quantity of stem cells, wherein the second quantity of stem cells is greater than the first quantity; administering a composition comprising, at least in part, said second quantity of stem cells; evaluating said at least one unique marker; determining whether said at least one unique marker is at an acceptable level; if said at least one unique marker is at an acceptable level, ending said method of treatment; and if said at least one unique marker is not at an acceptable level, waiting a period of time and repeating each of the determining, proliferating and administering steps.

Optionally, preparing the platelet rich plasma comprises: placing the patient's blood into a plurality of tubes; centrifuging the plurality of tubes for a predefined period of time to produce the platelet rich plasma; and aliquoting the produced platelet rich plasma into a sterile tube. Optionally, the method further comprises shaking the sterile tube after aliquoting.

Optionally, the method further comprises shaking the platelet rich plasma after treating with modulated pulses of laser light.

Optionally, the predefined wavelength ranges from 300 nm to 1000 nm.

Optionally, the predefined wavelength is in a range of 580 nm to 770 nm.

Optionally, the predefined period of time ranges from 1 minute to 5 minutes.

Optionally, the second quantity of stem cells is greater than the first quantity by 10% up to 400%.

Optionally, repeating each of the determining, proliferating and administering steps occurs 1 day to 6 months after said administering of the composition comprising, at least in part, said second quantity of stem cells.

Optionally, the marker is an ejection fraction percentage and said ejection fraction percentage increases by at least 8% after said administering of the composition comprising, at least in part, said second quantity of stem cells.

Optionally, the marker is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and said marker improves by at least 8% after said administering of the composition comprising, at least in part, said second quantity of stem cells.

Optionally, the disease comprises at least one of a cardiac disease, a neurodegenerative disease, a musculoskeletal trauma, a neurological trauma, type 2 diabetes, liver disease, lung disease, pancreatic disease, a psychosis disorder, or a psychiatric disorder.

The present specification also discloses a method of producing a composition that, when administered to a patient, treats a disease in a patient characterized by a predefined parameter, the method comprising: characterizing the predefined parameter for determining said parameter to be out of a predefined acceptable parameter range; obtaining platelet rich plasma comprising a first quantity of autologous hVSELs from the patient; increasing a number of said hVSEL stem cells from the first quantity to a second quantity by applying to said platelet rich plasma pulses of modulated laser light; harvesting the second quantity of hVSEL stem cells; administering the second quantity of hVSEL stem cells to the patient; reevaluating the patient's predefined parameter; determining if the patient's predefined parameter is at an acceptable level; repeating at predefined intervals of time each of the obtaining, increasing, harvesting, and administering steps if the patient's predefined parameter is not at the acceptable level; and concluding said method of treating the patient if the patient's predefined parameter is at the acceptable level.

Optionally, after performing each of the obtaining, increasing, harvesting, and administering steps, the patient's predefined parameter improves by at least 8%.

Optionally, the second quantity of hVSEL stem cells is greater than the first quantity by 10% up to 400%.

Optionally, said predefined intervals of time are in a range of 1 day to 6 months, or any time increment therein.

Optionally, the pulses of modulated laser light have a predefined wavelength in a range of 580 nm to 770 nm.

Optionally, the predefined parameter is an ejection fraction percentage and said ejection fraction percentage increases by at least 8% after said administering the second quantity of hVSEL stem cells.

Optionally, the predefined parameter is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and said predefined parameter improves by at least 8% after said administering the second quantity of hVSEL stem cells.

Optionally, the disease comprises at least one of a cardiac disease, a neurodegenerative disease, a musculoskeletal trauma, a neurological trauma, type 2 diabetes, liver disease, lung disease, pancreatic disease, a psychosis disorder, or a psychiatric disorder.

In some embodiments, the present specification is directed towards a method of treating a disease in a patient comprising: proliferating stem cells of the patient, wherein the proliferation comprises preparing platelet rich plasma containing stem cells and treating the platelet rich plasma containing stem cells with modulated pulses of laser light having a predefined wavelength and for a predefined period of time; and, administering the treated platelet rich plasma to the patient.

In some embodiments, the present specification discloses a method of treating a disease in a patient comprising: proliferating stem cells of the patient, comprising: adding normal human blood into a plurality of tubes; centrifuging the plurality of tubes at a predefined g force for 10 minutes to produce platelet rich plasma; shaking the plurality of tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma; and, administering the treated platelet rich plasma to the patient.

In some embodiments, the present specification describes a method of producing a composition that, when administered to a patient, treats a disease state of a patient comprising: proliferating stem cells of the patient, comprising: adding normal human blood into a plurality of tubes; centrifuging the plurality of tubes at a predefined g force for 10 minutes to produce platelet rich plasma; shaking the plurality of tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma.

In some embodiments, the present specification also discloses a method of treating a disease state of a patient comprising: proliferating stem cells of the patient, comprising: adding normal human peripheral blood into six tubes; centrifuging the six tubes at a predefined g force for 10 minutes to produce platelet rich plasma; shaking the six tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma.

In some embodiments, the present specification is directed towards a method of reducing biological age of a patient comprising: proliferating stem cells of the patient, wherein the proliferation comprises preparing platelet rich plasma containing stem cells and treating the platelet rich plasma containing stem cells with modulated pulses of laser light having a predefined wavelength and for a predefined period of time; and, administering the treated platelet rich plasma to the patient.

Optionally, the platelet rich plasma is prepared by: adding the patient's blood into a plurality of tubes; centrifuging the plurality of tubes at a predefined g force for a predefined period of time to produce the platelet rich plasma; and aliquoting the produced platelet rich plasma into a sterile tube.

Optionally, the centrifuging the plurality of tubes further comprises shaking the plurality of tubes after centrifuging.

Optionally, the method further comprises shaking the sterile tube after aliquoting.

Optionally, the plurality of tubes ranges from 3 tubes to 12 tubes, and any increment therein.

Optionally, the method further comprises shaking the platelet rich plasma after treating with modulated pulses of laser light.

Optionally, the treatment of the platelet rich plasma is carried out in minimum background white light conditions.

Optionally, the predefined wavelength ranges from 300 nm to 1000 nm. Still optionally, the predefined wavelength is 670 nm. Still optionally, the predefined wavelength is in a range of 580 nm to 770 nm.

Optionally, the platelet rich plasma is prepared using normal human blood.

Optionally, the predefined period of time ranges from 1 minute to 5 minutes.

Optionally, the treated platelet rich plasma has an amount of stem cells ranging from 0.5×6/mL to 2.0×10⁶/mL when analyzed immediately after the predefined period of time. Optionally, the treated platelet rich plasma has an amount of stem cells ranging from 0.5×10⁶ per mL to 2.0×10⁶ per mL when analyzed immediately after the predefined period of time.

Optionally, the patient experiences a decrease in biological age in a range of 1 year to 4 years based on a first administration of the treated platelet rich plasma. Still optionally, the patient experiences a decrease in biological age in a range of 4 years to 9 years based on a second administration of the treated platelet rich plasma. Optionally, the second administration of the treated platelet rich plasma occurs 1 week to 6 months after the first administration.

In some embodiments, the present specification discloses a method of reducing biological age of a patient comprising: proliferating stem cells of the patient, comprising: adding normal human blood into a plurality of tubes; centrifuging the plurality of tubes at a predefined g force for minutes to produce platelet rich plasma; shaking the plurality of tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma; and, administering the treated platelet rich plasma to the patient.

Optionally, the plurality of tubes ranges from 3 tubes to 12 tubes, and any increment therein.

Optionally, the treatment of the platelet rich plasma is carried out in minimum background white light conditions.

Optionally, the predefined wavelength ranges from 300 nm to 1000 nm. Still optionally, the predefined wavelength is 670 nm. Still optionally, the predefined wavelength is in a range of 580 nm to 770 nm.

Optionally, the predefined period of time ranges from 1 minute to 5 minutes.

Optionally, the treated platelet rich plasma has an amount of stem cells ranging from 0.5×6/mL to 2.0×10⁶/mL when analyzed immediately after the predefined period of time. Optionally, the treated platelet rich plasma exhibits a 2.5 fold increase in stem cells compared to a mean of first and second control samples, wherein the first control sample includes the platelet rich plasma treated with white torch light for the predefined period of time, and wherein the second control sample includes the platelet rich plasma without any light treatment.

Optionally, the modulation cancels a central wavelength band of the laser light such that the remaining upper and lower wavelength bands create a beat frequency pattern of sparse nodes.

Optionally, the patient experiences a decrease in biological age in a range of 1 year to 4 years based on a first administration of the treated platelet rich plasma. Still optionally, the patient experiences a decrease in biological age in a range of 4 years to 9 years based on a second administration of the treated platelet rich plasma. Optionally, the second administration of the treated platelet rich plasma occurs 1 week to 6 months after the first administration.

In some embodiments, the present specification discloses a method of producing a composition that, when administered to a patient, reduces a biological age of a patient comprising: proliferating stem cells of the patient comprising: preparing platelet rich plasma containing stem cells; and treating the platelet rich plasma with modulated pulses of laser light having a predefined wavelength and for a predefined period of time.

Optionally, the platelet rich plasma is prepared by: adding the patient's blood into a plurality of tubes; centrifuging the plurality of tubes at a predefined g force for a predefined period of time to produce the platelet rich plasma; and aliquoting the produced platelet rich plasma into a sterile tube.

Optionally, centrifuging the plurality of tubes further comprises shaking the plurality of tubes after centrifuging.

Optionally, the method further comprises shaking the sterile tube after aliquoting.

Optionally, the plurality of tubes ranges from 3 tubes to 12 tubes, and any increment therein.

Optionally, the method further comprises shaking the platelet rich plasma after treating with modulated pulses of laser light.

Optionally, the treatment of the platelet rich plasma is carried out in minimum background white light conditions.

Optionally, the predefined wavelength ranges from 300 nm to 1000 nm. Still optionally, the predefined wavelength is 670 nm. Still optionally, the predefined wavelength is in a range of 580 nm to 770 nm.

Optionally, the platelet rich plasma is prepared using normal human blood.

Optionally, the predefined period of time ranges from 1 minute to 5 minutes.

Optionally, the treated platelet rich plasma has an amount of stem cells ranging from 0.5×6/mL to 2.0×10⁶/mL when analyzed immediately after the predefined period of time.

In some embodiments, the present specification describes a method of producing a composition that, when administered to a patient, reduces a biological age of a patient comprising: proliferating stem cells of the patient, comprising: adding normal human blood into a plurality of tubes; centrifuging the plurality of tubes at a predefined g force for 10 minutes to produce platelet rich plasma; shaking the plurality of tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma.

Optionally, the plurality of tubes ranges from 3 tubes to 12 tubes, and any increment therein.

Optionally, the treatment of the platelet rich plasma is carried out in minimum background white light conditions.

Optionally, the predefined wavelength ranges from 300 nm to 1000 nm. Optionally, the predefined wavelength is 670 nm.

Optionally, the predefined period of time ranges from 1 minute to 5 minutes.

Optionally, the treated platelet rich plasma has an amount of stem cells ranging from 0.5×6/mL to 2.0×10⁶/mL when analyzed immediately after the predefined period of time.

Optionally, the treated platelet rich plasma exhibits a 2.5 fold increase in stem cells compared to a mean of first and second control samples, wherein the first control sample includes the platelet rich plasma treated with white torch light for the predefined period of time, and wherein the second control sample includes the platelet rich plasma without any light treatment.

Optionally, the modulation cancels a central wavelength band of the laser light such that the remaining upper and lower wavelength bands create a beat frequency pattern of sparse nodes.

In some embodiments, the present specification discloses a method of reducing biological age of a patient comprising: proliferating stem cells of the patient comprising: preparing platelet rich plasma containing stem cells; and treating the platelet rich plasma with modulated pulses of laser light having a predefined wavelength and for a predefined period of time.

Optionally, the platelet rich plasma is prepared by: adding the patient's blood into six tubes; centrifuging the six tubes at a predefined g force for a predefined period of time to produce the platelet rich plasma; and aliquoting the produced platelet rich plasma into a sterile tube. Optionally, centrifuging the six tubes further comprises shaking the six tubes after centrifuging. Optionally, the method further comprises shaking the sterile tube after aliquoting.

Optionally, the method further comprises shaking the platelet rich plasma after treating with modulated pulses of laser light.

Optionally, said treatment of the platelet rich plasma is carried out in minimum background white light conditions.

Optionally, the predefined wavelength ranges from 300 nm to 1000 nm.

Optionally, the predefined wavelength is 670 nm.

Optionally, the platelet rich plasma is prepared using normal human peripheral blood.

Optionally, the predefined period of time is 3 minutes.

Optionally, said treated platelet rich plasma has 1.256×10⁶/mL of stem cells when analyzed immediately after the predefined period of time.

In some embodiments, the present specification also discloses a method of reducing biological age of a patient comprising: proliferating stem cells of the patient, comprising: adding normal human peripheral blood into six tubes; centrifuging the six tubes at a predefined g force for 10 minutes to produce platelet rich plasma; shaking the six tubes; aliquoting the produced platelet rich plasma into a sterile tube; shaking the platelet rich plasma in the sterile tube; treating the platelet rich plasma with modulated pulses of laser light having a predefined wavelength and for a predefined period of time; and shaking the treated platelet rich plasma.

Optionally, said treatment of the platelet rich plasma is carried out in minimum background white light conditions.

Optionally, the predefined wavelength ranges from 300 nm to 1000 nm.

Optionally, the predefined wavelength is 670 nm.

Optionally, the predefined period of time is 3 minutes.

Optionally, said treated platelet rich plasma has 1.256×10⁶/mL of stem cells when analyzed immediately after the predefined period of time.

Optionally, said treated platelet rich plasma exhibits a 2.5 times increase in stem cells compared to a mean of first and second control samples, wherein the first control sample includes the platelet rich plasma treated with white torch light for the predefined period of time, and wherein the second control sample includes the platelet rich plasma without any light treatment.

Optionally, said modulation cancels a central wavelength band of the laser light such that the remaining upper and lower wavelength bands create a beat frequency pattern of sparse nodes.

In some embodiments, the present specification is directed toward a method of proliferating stem cells comprising: preparing platelet rich plasma containing stem cells; and treating the platelet rich plasma with modulated pulses of laser light having a predefined wavelength and for a predefined period of time.

Optionally, the platelet rich plasma is prepared by: adding donated normal human peripheral blood into three tubes; centrifuging the three tubes at a predefined g force for a predefined period of time to produce the platelet rich plasma; and aliquoting the produced platelet rich plasma into a single sterile tube.

Optionally, said treatment of the platelet rich plasma is carried out in minimum background white light conditions.

Optionally, the predefined wavelength ranges from 300 nm to 1000 nm.

Optionally, the predefined wavelength is 670 nm.

Optionally, the platelet rich plasma is prepared using donated normal human peripheral blood.

Optionally, the predefined period of time is 3 minutes.

Optionally, said treated platelet rich plasma has 1.256×10⁶/mL of stem cells when analyzed immediately after the predefined period of time.

In some embodiments, the present specification discloses a method of proliferating stem cells comprising: adding donated normal human peripheral blood into three tubes; centrifuging the three tubes at a predefined g force for 10 minutes to produce platelet rich plasma; aliquoting the produced platelet rich plasma into a single sterile tube; and treating the platelet rich plasma with modulated pulses of laser light having a predefined wavelength and for a predefined period of time.

Optionally, said treatment of the platelet rich plasma is carried out in minimum background white light conditions.

Optionally, the predefined wavelength ranges from 300 nm to 1000 nm.

Optionally, the predefined wavelength is 670 nm.

Optionally, the predefined period of time ranges from 1 to 6 minutes. Optionally, the predefined period of time ranges from 1 to 3 minutes. Optionally, the predefined period of time is 3 minutes. Optionally, the predefined period of time is dependent upon the volume of platelet rich plasma.

Optionally, said treated platelet rich plasma has 1.256×10⁶/mL of stem cells when analyzed immediately after the predefined period of time.

Optionally, said treated platelet rich plasma exhibits a 2.5 times increase in stem cells compared to a mean of first and second control samples, wherein the first control sample includes the platelet rich plasma treated with white torch light for the predefined period of time, and wherein the second control sample includes the platelet rich plasma without any light treatment.

Optionally, said modulation cancels a central wavelength band of the laser light such that the remaining upper and lower wavelength bands create a beat frequency pattern of sparse nodes.

The aforementioned and other embodiments of the present shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates a Strachan-Ovokaitys Node Generator (SONG) device as disclosed in U.S. Pat. No. 6,811,564, which is incorporated herein by reference in its entirety;

FIG. 2 shows a sparse constructive interference effect from a 1 percent bandwidth cancellation plate having a 5 mm aperture;

FIG. 3 is a flowchart illustrating steps of a method of preparing PRP containing hVSEL stem cells, in accordance with some embodiments of the present specification;

FIG. 4 is a graph showing data on the numbers and distribution of hVSEL stem cells in untreated PRP;

FIG. 5 is a graph illustrating data pertaining to Costa laser+SONG modulation of PRP, related controls and in vitro culture for one day;

FIG. 6 is a graph illustrating data pertaining to Magna Costa Laser exposure time variation and SONG modulation variation on Day 0 and Day 5;

FIG. 7 is a graph illustrating data pertaining to Costa Laser treatment of hVSEL stem cells in PRP at Day 0, Day 1, and Day 7;

FIG. 8 is a graph illustrating data pertaining to time titration of SONG modulated Magna Costa and Costa Laser on hVSEL Stem Cells in PRP;

FIG. 9A illustrates an intrinsic epigenetic age (IEA) of two patients;

FIG. 9B illustrates an IEA of another two patients;

FIG. 9C illustrates an IEA of yet another two patients;

FIG. 9D illustrates an IEA of yet another two patients;

FIG. 10 provides an exemplary process of preparing PRP that contains hVSEL stem cells, obtained from blood samples of a patient, for treatment of various indications such as end stage heart failure or reduction of IEA, in accordance with some embodiments of the present specification;

FIG. 11 illustrates a method of harvesting and storing hVSEL stem cells for subsequent use, in accordance with an embodiment of the present specification;

FIG. 12 is a flowchart illustrating a method of treating end stage heart failure, in accordance with an embodiment of the present specification; and

FIG. 13 is a flowchart illustrating a method of treating cardiac disease, whereby the disease state is characterized by a low ejection fraction, in accordance with an embodiment of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

In embodiments, intrinsic epigenetic age (IEA) refers to a true biological age at the DNA level. In embodiments, extrinsic epigenetic age (EEA) refers to an organism's immune function status in addition to other factors that are more responsive to external factors such as diet, lifestyle and supplement use.

In embodiments, “normal human blood” is defined as blood in a chemical and physical state as when immediately withdrawn from a human and without any further processing, whether mechanical and/or chemical, also referred to as non-processed human blood. As used in this specification, peripheral blood is the fluid that travels through the heart, arteries, capillaries, and veins. It serves to transport oxygen and other nutrients to the body's cells and tissues and to remove carbon dioxide and other waste products from the body. Peripheral blood also plays an essential role in the immune system, delivery of hormones, and temperature regulation.

Platelet-rich plasma (PRP) may be defined, as used in this specification, as plasma that has a heightened amount of platelets due to some form of mechanical and/or chemical processing relative to plasma that has not undergone that processing.

Nano-Bioelectronic Photo-Acoustic Therapy (NPT), may be defined, as used in this specification as use of a SONG modulated laser to activate hVSEL stem cells in PRP. NPT includes SONG modulated laser being applied to stem cells in vitro and directly to the patient.

### SONG Device

SONG modulated laser light may remove blocking proteins from hVSEL stem cell surface antigens and thus induce the biological processes required for the hVSEL stem cells to have maximum effectiveness in tissue repair and regeneration.

In various embodiments, for increased production or proliferation, the stem cells are treated with a laser process that exposes the stem cells to a predefined laser wavelength at a predefined amplitude modulation that is passed through a beam expander, typically on the order of 5× to 10×, (though greater or lesser could be used) and in conjunction with a device for optical phase conjugation such as a Strachan-Ovokaitys Node Generator or SONG device, which is disclosed in U.S. Pat. No. 6,811,564 and incorporated herein by reference.

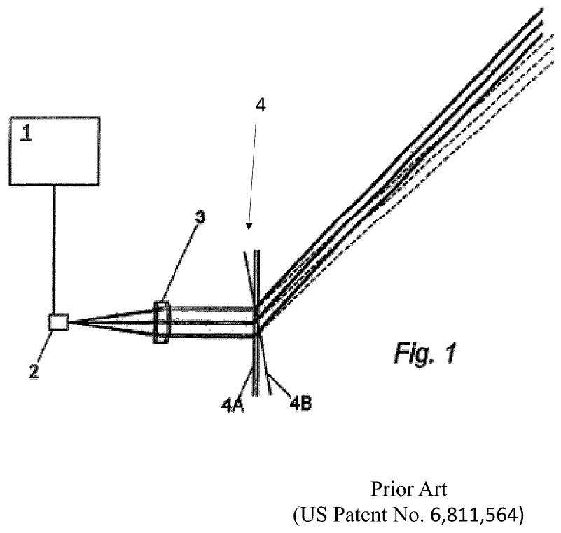

FIG. 1 illustrates a SONG device as disclosed in U.S. Pat. No. 6,811,564. Referring to FIG. 1, the SONG device comprises a laser diode 2 which is controlled by an amplitude modulator 1. The laser diode 2 is selected to have a substantially linear relationship between current and wavelength with minimum mode hopping. The amplitude modulator 1 modulates the current to the laser diode 2 which, in turn, results in a very small wavelength modulation of the laser, for purposes discussed below.

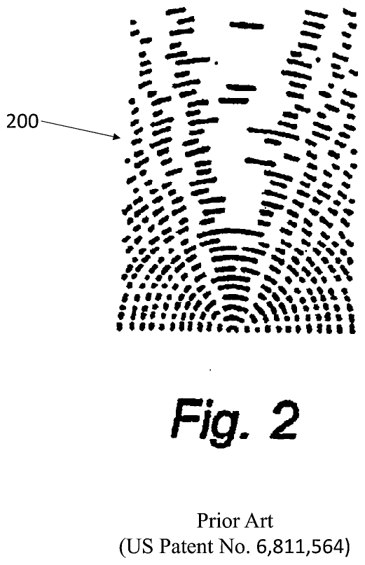

The output of the laser diode 2 is collimated by a lens 3 and passed to an optical element 4. The optical element 4 consists of a first diffraction grating, a refractive element, and a second diffraction grating such that the beam is substantially cancelled. This allows the cancellation to occur over a small percentage of the wavelength variance of the laser source, rather than at a single critical wavelength. Wavelengths beyond the acceptance bandwidth of the cancelling optic 4 above and below the center frequency pass without being cancelled. This means that a complex Fresnel/Fraunhoffer zone is generated, defined by the beat frequency of the high and low frequencies as a function of the aperture. Consequently, relatively sparse zones of constructive interference occur between the high and low frequency passes of the cancellation element in selected directions from the aperture, as shown in FIG. 2. FIG. 2 shows the sparse constructive interference effect from a 1 percent bandwidth cancellation plate of 5 mm aperture. Black represents constructive nodes.

As seen in FIG. 1, the optical element 4 can be adjusted angularly between positions 4A and 4B. This varies the ratio of constructive to destructive interference. Additionally, in embodiments, the system of FIG. 1 may include mechanisms for aligning the resultant beam emerging from optical element 4, in a straight line with the collimated beam emerging from collimator 3.

In effect, the continuous beam is transformed into a string of extremely short duration pulses typically on the order of a duration in subfemtoseconds. The small wavelength modulation of the laser diode 2 causes the constructive and destructive nodes to move rapidly through the volume of the Fresnel zone of the collimator lens aperture. This has the effect of stimulating very short (subpicosecond) pulse behavior at any point in the Fresnel zone through which the nodes pass at a pulse repetition frequency defined by the amplitude modulator frequency.

The wavelength of the cancellation and constructive interference zones for a theoretical single path would be the difference between the two frequencies. If the bandwidth of the cancelling element is narrow, this difference is very small and the effective wavelength of the cancelled/non-cancelled cycle would be very long, on the order of pico-seconds. Therefore, the system would behave substantially similarly to a system with no cancellation because it requires an aperture much larger than the primary light wavelength to generate a useful Fresnel/Fraunhoffer zone. Such an aperture would greatly multiply the available Feynman diagram paths eliminating any useful effect, even if it were possible to generate a sufficiently coherent source of such an aperture.

If the beat frequency can be made high enough, the wavelength of the cancelled to non-cancelled cycle can be a fraction of a practical aperture. This will make this wavelength sufficiently small to limit the Feynman paths to within a cycle or two in free space allowing the Fresnel/Fraunhoffer effect to be apparent. Since the center frequency and spectrum spread of a laser diode is modulated by adjusting the current and/or temperature of the junction, the pattern of the Fresnel/Fraunhoffer zones is varied substantially by very small variations in the wavelength of one or both pass frequencies. Such modulation is produced in the apparatus of FIG. 1 by the amplitude modulator 2.

A conventional coherent or incoherent beam would have high probability paths in the Feynman diagram. These paths would overlap at very low frequencies (kHz) and be of little practical use in the stimulation of molecular resonance. It should be noted however that the phenomena described above is used as a means to multiply the modulation frequency, up to the point where the beam effectively becomes continuous. Thus, by properly selecting the aperture, the region of the beam selected for transmission through the medium, and the modulation frequency, it is possible to cause the constructive nodes to pass across any given point in the beam at frequencies many times higher than the modulation frequency. In ideal conditions, the duration of exposure to a constructive node of any point would be for a period equivalent to a quarter of the duration of a wavelength of the molecular frequency repeated once per cycle.

If the wavelength of the laser is chosen to be one easily absorbed by the atomic structures it is desired to induce to resonance, then the beam will efficiently deliver the desired modulation frequency to the desired molecules. Cell adhesion molecules and human integrins such as alpha 4 and beta 1 are ideally suited for excitation to chemical activity by this method.

The sources of cells for the procedure described herein may be autologous or exogenous. Autologous stem cells refer to cells which are derived from the same person who is to be treated with such cells. These cells will be a genetic match obviating risks of rejection of cells. In current methods, autologous stem cells are either derived or concentrated from peripheral blood, bone marrow or fat, yet other tissues could be a source of autologous stem cells as virtually every tissue of the body has its own distinct stem cell reservoir.

A preferred exogenous source of stem cells is umbilical cord blood. Stem cells from cord blood are very robust with long telomeres (a genetic aging clock level of newborn level) and a strong capacity for tissue repair. Functionally, rejection syndromes of the cells and graft versus host disease (GVHD) have not been an issue with this source of cells in the context of an intact immune system. Matched bone marrow could also be a source of cells, though a high degree of matching would be required to avoid rejection and GVHD. In practice, for regeneration as opposed to anti-leukemic medical regimes, cord blood stem cells have been used safely.

Collection of hVSEL Stem Cells for SONG Modulated Laser Activation

hVSEL stem cells are known to persist throughout all stages of life, suggesting a potential homeostatic mechanism which maintains the hVSEL stem cell pool. There is a constant availability of hVSEL stem cells in the PRP of patients who have undergone multiple PRP collections. The bone marrow is the likely source of the hVSEL stem cells for maintaining the peripheral blood hVSEL stem cell pool. This is supported by the mobilization of hVSEL stem cells into the peripheral blood following acute myocardial infarction. In further support of this concept, hVSEL stem cells have been shown to be present in human bone marrow itself and also in human leukapheresis products. Therefore, hVSEL stem cells migrate from the bone marrow to the peripheral blood during physiological homeostasis and during pathological stimuli.

There is a population of quiescent VSEL stem cells in murine bone marrow which may be resistant to extrinsic heat stress in the same way as the quiescent population of MSC derived from desquamated endometrium of menstrual blood (eMSC). This raises the possibility that quiescent hVSEL stem cells residing in the bone marrow may be available for collection and SONG modulated laser activation even after high-dose chemotherapy. Such an approach has the ability to enhance somatic cell and tissue repair following high dose chemotherapy and may even indicate a possible benefit of elective hVSEL stem cells harvest, SONG modulated laser activation, and cryopreservation for later therapeutic application to restore somatic cells use following high dose chemotherapy.

Preparation of Platelet Rich Plasma (PRP) Containing hVSEL Stem Cells

Embodiments of the present specification use SONG modulated laser light to interact with hVSEL in PRP to upregulate the expression of CXCR4, Oct 3/4 and SSEA4. It should be appreciated that CXCR4 is C-X-C chemokine receptor 4 or fusin, which is a protein on the surface of certain immune system cells, including CD4 T lymphocytes (CD4 cells), Oct 3/4 is a regulatory gene that maintains the pluripotency and self-renewal properties of embryonic stem cells (ESCs), and SSEA4 is a stage specific glycolipid cell surface antigen involved in cellular signal transduction, cell recognition and cell adhesion which are important in pluripotent stem cell biology.

This is important, for example, in terms of cell locomotion, chemotaxis, signaling, and adhesion where CXCR4 expression is increased in SONG modulated laser activated hVSEL stem cells. Further, the increased expression of Oct 3/4 in response to the SONG modulated laser is not only a marker of pluripotent stem cells, but it is also important in driving cell differentiation towards the cardiac lineage and to the development of the mesoderm from the embryonic epiblast. More recently, Oct 3/4 has been further implicated in the reprogramming of somatic cells making increased expression desirable in regenerative medicine protocols.

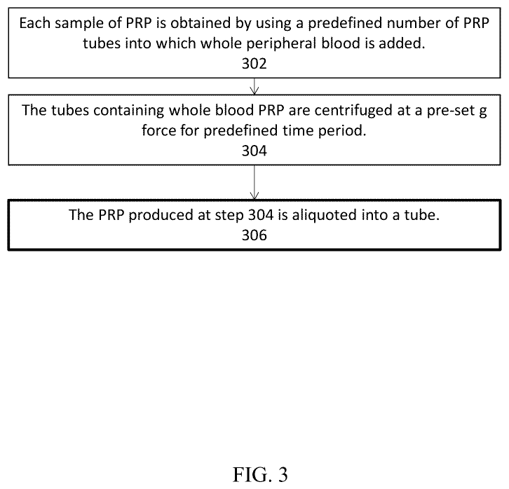

FIG. 3 is a flowchart illustrating a method of preparing PRP that contains hVSEL stem cells, in accordance with some embodiments of the present specification. Anti-coagulated (sodium citrate) donated normal human peripheral blood (450 mL) was acquired and kept at 4° C. before use. The blood was allowed to warm to room temperature before processing for PRP.

At step 302, each sample of PRP is obtained by using three PRP tubes into which 11 mL of whole peripheral blood (normal human blood) is added/aliquoted. At step 304, the tubes containing whole blood PRP are centrifuged at a pre-set g force for 10 minutes. Consequently, each of the three PRP tubes containing 11 mL of whole blood produces approximately 6 mL (a total of approximately 18 mL) of PRP.

At step 306, the PRP produced at step 304 is aliquoted, using aseptic technique in a Class II flow hood, into a single sterile tube for further manipulation and analysis. Each 18 mL PRP preparation is created in triplicate for each manipulation and assessment process.

Processing, Modulation, Manipulation and Assessment of Human PRP Containing hVSEL Stem Cells

In general, the application of NPT to autologous stem cells has a wide-ranging impact in the treatment of diseases including cardiac disease, neurodegenerative disease, neurological trauma, type 2 diabetes, liver disease, lung disease, pancreatic disease, and psychosis and psychiatric disorders. The pluripotent nature of hVSEL stem cells suggests that they may be useful throughout the body as a therapeutic procedure. One of the applications described subsequently herein include modulation of the aging process. Another application describes use in treatment of end stage heart failure.

In accordance with aspects of the present specification, PRP containing hVSEL stem cells are manipulated or modified using pulses of laser light having a wavelength in a range of 300 nm to 1000 nm, and, in an embodiment, the predefined wavelength is in a range of 580 nm to 770 nm, or preferably 670 nm. In some embodiments, PRP containing hVSEL stem cells is manipulated or modified using the following two lasers:

Costa Laser: The Costa Laser employed is, in an embodiment, a 670 nm, 5 mW SONG modulated laser. In embodiments, the level of optical phase conjugation (OPC) was varied for experimental purposes. In embodiments, a level of optical phase conjugation ranges from 1% to 99%. In an embodiment, the SONG modulated laser was set at 60% optical phase conjugation (OPC) for a resultant beam power of 1 mW.
Magna Costa Laser: The Magna Costa laser employed is, in an embodiment, a 670 nm, 5.7 mW SONG modulated laser. In embodiments, the level of optical phase conjugation (OPC) was varied for experimental purposes. In embodiments, a level of optical phase conjugation ranges from 1% to 99%. In an embodiment, the SONG modulated laser was set at 60% OPC for a resultant beam power of 1.3 mW. The Magna Costa laser has adjustable wave forms to enable alternative wave forms to be introduced as a control.

It should be appreciated that, while the examples provided herein may refer to the use of a Costa Laser or Magna Costa Laser, these laser systems are exemplary only. Any modulated laser beam in accordance with the laser systems described in the patent publications incorporated by reference may be used.

In embodiments, SONG modulation of the laser cancels the central wavelength band of the laser output as a result of non-fringing destructive interference. The remaining upper and lower wavelength bands create a beat frequency pattern of sparse nodes of constructive interference which represents the physical visible light that remains. Modulation of this complex wave form pattern results in a rapid traverse of these nodes that can reach pulse repetition frequencies every femtosecond or less. The destructive interference and sparseness of the nodes reduces the flare at the surface of the tissue interface. This decreases both the reflectiveness of photons which have entered a zone that has just experienced photon absorption as well as a scattering effect. The depth of penetration of sparse nodes may be 10-20 times that of ordinary photons at the surface of an interface such as human skin.

Culture and Harvesting of Laser-Treated and Control (No Laser or White Light) hVSEL in PRP

In some embodiments, to assess the biological stability of the effect of laser exposure or manipulation, the PRP is cultured in equal volumes of RPMI 1640 media supplemented with 200 mM L-Glutamine, penicillin, and streptomycin and 10% heat inactivated fetal calf serum.

All PRP cultures are carried out using T25 vented flasks in a humidified incubator set at 37° C. and 5% CO₂ in air. Adherent cells are harvested when needed in an initial wash with Ca2+/Mg2+ free Dulbecco's PBS and treatment with Trypsin EDTA for 5 minutes at 37° C.

The Numbers and Distribution of hVSEL Stem Cells in Untreated PRP

In an embodiment, to assess the distribution and numbers of hVSEL stem cells in untreated PRP tubes of PRP are separated into discrete sample tubes following centrifugation (see the flowchart of FIG. 3) by taking 2 mL of the ‘top’ portion of PRP; 2 mL of the ‘middle’ portion of PRP; 2 mL of the ‘bottom’ portion PRP—as close to the red cell interface as possible; 2 mL of the top of the red cell section; and 2 mL at the bottom of the red cell section for a total of five tubes per PRP sample. Each sample is assessed for hVSEL stem cell numbers using the flow cytometry protocol mentioned earlier in this specification. Assessment of each sample showed that cell viability remained at >90%.

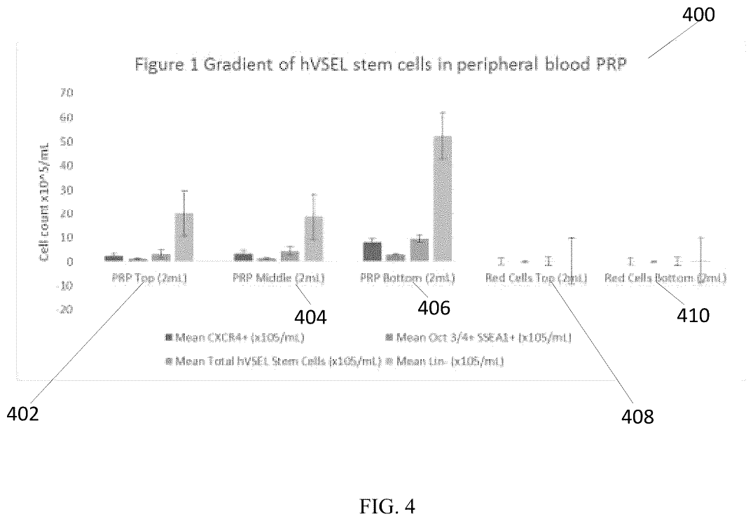

FIG. 4 is a graph 400 illustrating data on the number and distribution of hVSEL stem cells in untreated PRP for each of the portions obtained in the method described with respect to FIG. 3. The top 2 mL 402 of the PRP is found to have a mean hVSEL stem cell count of 3.1×10⁵/mL and the mean Lin− cell count was 20.0×10⁵/mL. The middle 2 mL 404 of the PRP is found to have a mean hVSEL stem cell count of 4.27×10⁵/mL and the mean Lin− cell count is 18.5×10⁵/mL. The bottom 2 mL 406 of the PRP is found to have a mean hVSEL stem cell count of 9.29×10⁵/mL and the mean Lin− cell count is 52.2×10⁵/mL. The total mean number of hVSEL stem cells found in PRP is 1.66×10⁶/mL. The total mean number of Lin− cells found in the PRP is 9.01×10⁶/mL. The total number of hVSEL stem cells in the red cell top section 408 is 4.0×10²/mL and the mean Lin− cell count is 1.65×10⁴/mL. The total number of hVSEL stem cells in the red cell bottom section 410 is 6.0>10²/mL and the mean Lin− cell count is 5.1×10⁴/mL.

In this embodiment, it is shown that there is a mean of 1.6×10⁶/mL hVSEL stem cells in PRP obtained from donated human blood. It is also possible to provide another mean estimate of the total hVSEL stem cells/mL in PRP by taking the mean of the no treatment values for PRP across all of the different extractions. This produces a mean value of 3.92×10⁶/mL. The range of observed hVSEL stem cells in PRP normal peripheral blood (normal human blood) in the present gradient study is 0.746−16×10⁵/mL.

The PRP is found to have a gradient of hVSEL stem cells increasing from the top meniscus of the PRP all the way down to the PRP/red cell interface where the highest number of hVSEL stem cells is found, indicating that the entire volume of PRP should be used for optimal results in some embodiments. Accordingly, multiple volumes of PRP are pooled for clinical applications. In embodiments, some applications may require a higher hVSEL concentration per volume of PRP. For example, in some applications, such as hair and cosmetic applications, which are localized, it is desirable to have a higher hVSEL concentration. In these cases, the bottom third portion where the highest number of hVSEL cells is present may be used for a more concentrated effect. In other applications, for example, in systemic treatment that may be administered intravenously, it may be desirable to pool the entire volume. There are very few (approximately 1×10³/mL) hVSEL stem cells in the red cell section of the PRP tube. These data show that the PRP based isolation of hVSEL stem cells works very efficiently when using the systems and methods of the present specification.

Laser Treatment (using Costa Laser) of PRP and Resulting hVSEL Stem Cell Proliferation on Day 0 and Day 1 of Culture

In an embodiment, to assess the effect of a Costa laser with SONG modulation on hVSEL stem cell numbers in PRP, PRP is prepared as described earlier with reference to FIG. 3, in triplicate. A first batch is exposed to Costa laser +SONG (set at 60% OPC)) light for 3 minutes, a second batch is exposed to white torch light for 3 minutes, and a third batch received no treatment (control). Following flow cytometer analysis, the three PRP samples are cultured and then harvested for flow cytometry analysis on day 1. The purpose of this embodiment is to assess the initial effects of the laser on hVSEL stem proliferation and to see if these changes were stable after 24 hours in culture in vitro. Others have described gene upregulation in human dermal cells following laser exposure which resulted in increased paracrine secretions.

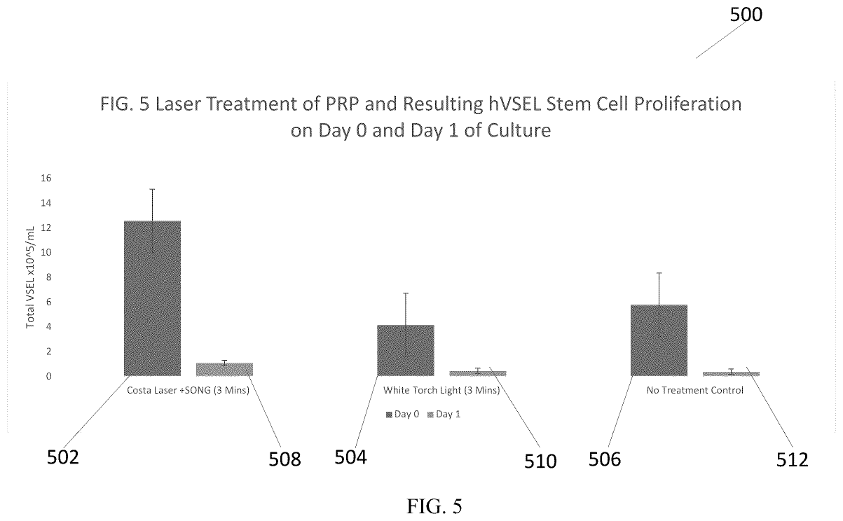

FIG. 5 is a graph 500 illustrating data on Costa laser +SONG modulation of PRP, related controls, and in vitro culture for a duration of one day. As shown, when PRP is treated with laser light +SONG 502 for 3 minutes, and analyzed by flow cytometry immediately afterwards, the number of hVSEL stem cells are 1.256×10⁶/mL. The same batch of PRP treated with white torch light 504 for 3 minutes (as a first control) and analyzed immediately contains 4.15×10⁵/mL hVSEL stem cells. The same batch of PRP, undergoing no treatment 506 (as a second control), contains 5.77×10⁵/mL hVSEL stem cells. The mean of these two control samples is 4.96×10⁵/mL. The laser exposed PRP therefore showed a 2.5× (2.5 fold) increase in hVSEL stem cell numbers compared to the mean of the two control groups. This is a rapid effect in that following modulated laser exposure the cells are taken immediately for analysis on the flow cytometer. The time from modulated laser exposure to flow cytometry analysis is therefore no greater than 30 minutes in any of the studies. This observation compares favorably with the clinical use and clinical trial of modulated laser exposed hVSEL in PRP which often show rapid clinical improvements following intravenous infusion of autologous laser exposed hVSEL stem cells in PRP. This is the first time that these laboratory observations and clinical data have been correlated.

In embodiments, it should be noted that the PRP may be treated with laser light +SONG for a predefined time period ranging from 1 minute to 5 minutes, and preferably 3 minutes. In embodiments, the treated platelet rich plasma has an amount of stem cells ranging from 0.5×10⁶/mL to 2.0×10⁶/mL when analyzed immediately after the predefined period of time. In embodiments, the number of hVSEL stem cells after treatment is are 1.256×10⁶/mL.

Therefore, it should be appreciated that platelet rich plasma having a first quantity of hVSEL stem cells is exposed to a laser treatment, as described herein, for a period of time ranging from 10 seconds to 10 minutes, preferably 1 minute to 5 minutes. After exposure, the platelet rich plasma has a second quantity of hVSEL stem cells wherein the second quantity is greater than the first quantity. In one embodiment, the second quantity is 10% to 400% greater than the first quantity.

On day 1 of culture in vitro (that is, following 24 hours culture in vitro), the Costa laser+SONG 508 treated PRP contains 1.086×10⁵/mL hVSEL stem cells. On day 1 of culture in vitro, the White Torch light PRP 510 (a first control) contained 0.448×10⁵/mL hVSEL stem cells.

On day 1 of culture in vitro the Control PRP 512 (no treatment and a second control) contained 0.376×10⁵/mL hVSEL stem cells. The mean of these two control groups is 0.432×10⁵/mL. The laser exposed PRP after 24 hours in vitro showed a 2.5× (2.5-fold) increase of hVSEL stem cells over the control cells indicating that even though the actual cell counts decreased (which is to be expected following culture in vitro), the ratio of laser modulated hVSEL stem cells to control hVSEL stem cells remained the same over 24 hours. In embodiments, stem cell administration occurs in a time frame ranging from within 1 minute to 24 hours of preparation/laser modulation. In embodiments, stem cell administration occurs in a time frame ranging from within 1 minute to 2 hours of preparation/laser modulation. In embodiments, stem cell administration preferably occurs within 30 minutes of preparation/laser modulation.

These data have confirmed that the laser has a proliferative effect on hVSEL stem cells in PRP. This effect is maintained in relative terms for at least 24 hours in vitro post laser exposure. In embodiments, stem cell administration may occur in a time frame wherein the measurable effect on hVSEL remains post laser exposure, wherein said time may vary depending on a plurality of conditions.

Laser Treatment (using Magna Costa Laser) of hVSEL Stem Cells in PRP With Titration of Laser Exposure Time and ±SONG Modulation at Day 0 and Day 5

In an embodiment, the hVSEL stem cells in PRP were treated with the Magna Costa laser to assess the numbers of hVSEL present in PRP following laser exposure from 1-3 minutes with and without the SONG modulation in order to confirm optimum settings for clinical use. As described earlier in this specification, the Magna Costa laser is the same as the Costa except for an adjustable wave form. This enables the use of a possibly improved control of a ‘flat’ wave in these experiments.

In this embodiment, the SONG modulation was set at 60% OPC and all cells are analyzed at Day 0 and then cultured in vitro for 5 days to assess the persistence of any proliferative changes in hVSEL.

The purpose of this embodiment is to assess the laser exposure time and the application of SONG modulation, or no SONG modulation, on the proliferation of hVSEL stem cells in PRP on the day of laser exposure (D0) and after five days in vitro (D5). The laser exposure times and SONG modulation are critical to successful hVSEL stem cell proliferation.

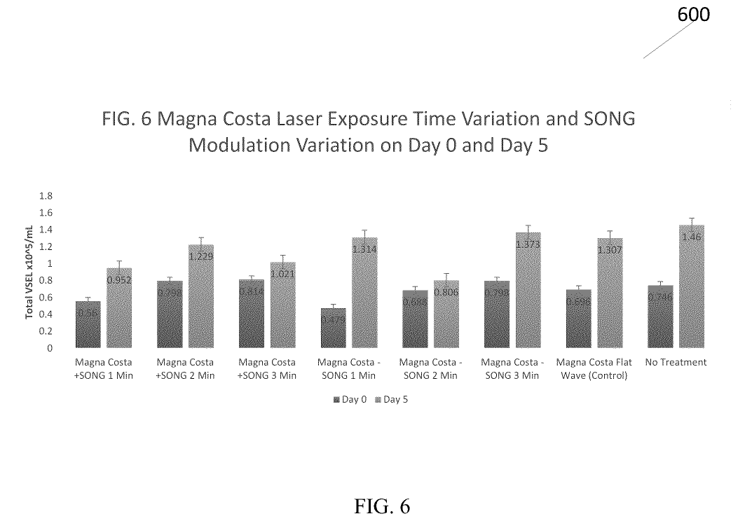

FIG. 6 is a graph 600 illustrating data pertaining to Magna Costa Laser exposure time variation and SONG modulation variation on day 0 and day 5. In embodiments, the laser exposure time ranges from 1 minute to 6 minutes. In embodiments, the laser exposure time ranges from 1 minute to 3 minutes. In embodiments, for a volume ranging from 20 milliliters to 30 milliliters, the laser exposure time is 3 minutes. In other embodiments, laser exposure time is dependent on the volume of PRP. In other embodiments, laser exposure time is dependent on the quality of harvested PRP.

As shown, on day 0 (the day when the PRP was prepared and lasered) the total number of hVSEL stem cells in the PRP increased as the laser exposure time was increased (from 1 minute to 3 minutes) and the SONG modulation was present throughout. The 2-minute and 3-minute laser exposure time produced very similar numbers of hVSEL stem cells. There was a similar but less pronounced rise in hVSEL stem cell numbers when the laser was applied without SONG modulation. The flat wave and no treatment controls remained similar, noting that the flat wave laser exposure time was 3 minutes.

Thus, in the PRP exposed to the SONG modulated Magna Costa laser for 1-minute, 2-minutes and 3-minutes the numbers of hVSEL stem cells are highest in the 2-minute and 3-minute treatments. In the Magna Costa laser without SONG modulation there are fewer hVSEL stem cells than in the laser SONG modulated group over 1, 2 and 3 minutes but there is a steady increase in detected hVSEL stem cells across the laser exposure times. The SONG modulated Magna Costa flat wave and no treatment controls (hVSEL numbers) are lower than the equivalent SONG modulated laser cell counts at 2-minute and 3-minute laser exposure.

On day 5 of culture in vitro the SONG modulated laser group show increased numbers of hVSEL stem cells compared to Day 0 with slightly more hVSEL stem cells present in the 2-minute and 3-minute laser exposure time. The 1-minute and 3-minute laser exposure without SONG modulation contains more hVSEL stem cells than the 2-minute laser exposure and the flat wave and no treatment controls also contain more hVSEL stem cells overall than in Day 0.

Thus, the day 5 hVSEL stem cell counts, after 5 days culture in vitro, all showed an increase in hVSEL stem cells compared to Day 0. There is also an increase in the control groups which appeared greater than the experimental groups. This anomaly needs further investigation because it could be a true reflection of in vitro proliferation of hVSEL stem cells or it may just be an anomaly in this particular embodiment. In general terms when lasered hVSEL stem cells are cultured in vitro then a reduction in cell numbers is observed.

Costa Laser Treatment (±SONG Modulation) of hVSEL stem cells in PRP at Day 0, Day 1 and Day 7

In an embodiment, the PRP (prepared in accordance with the method of FIG. 3) is exposed to the Costa laser for 3 minutes with SONG modulation and 3 minutes without SONG modulation. The resultant PRP is then assessed for hVSEL proliferation and then put into in vitro culture for 1 and 7 days. Cultures are harvested on Day 1 and Day 7 and the resultant cell harvest is assessed for hVSEL proliferation using flow cytometry. This embodiment also includes an assessment of hVSEL numbers in whole peripheral blood following red cell lysis.

This embodiment is directed towards assessing the numbers of hVSEL stem cells in PRP on the day of laser treatment and at Day 1 and Day 7 culture of the cells in vitro and to assess the effect of laser treatment with and without SONG modulation. A measurement is made on the number of hVSEL stem cells in peripheral blood without any treatment. This involved red cell analysis followed by flow cytometry.

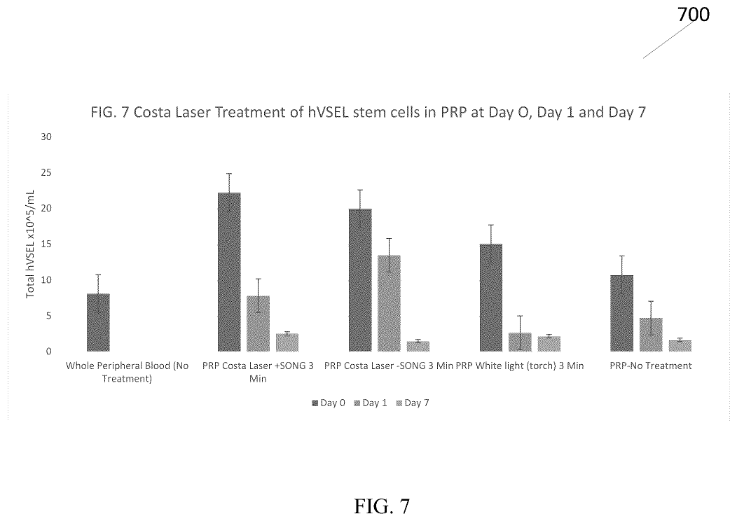

FIG. 7 shows a graph 700 illustrating data pertaining to Costa Laser treatment of hVSEL stem cells in PRP at Day 0, Day 1 and Day 7. The number of hVSEL stem cells in this sample of peripheral blood is 8.1×10⁵/mL which correlates well with previous estimates of hVSEL stem cells in PRP at 1×10⁶/mL and the hVSEL stem cells in PRP control in this study of 1.072×10⁶/mL. It is to be expected that PRP will have slightly higher hVSEL stem cell counts than peripheral blood as hVSEL stem cells are concentrated in PRP.

The number of hVSEL stem cells in PRP following 3 minutes of SONG modulated laser treatment is increased to 2.22×10⁶/mL, on Day 1 of culture it is 7.82×10⁵/mL and on day 7 of culture it is 2.56×10⁵/mL. The number of hVSEL stem cells in PRP following 3 minutes of unmodulated laser treatment is increased to 1.994×10⁶/mL, on Day 1 of culture it is 1.348×10⁶/mL and on day 7 of culture it is 1.48×10⁵/mL.

The number of hVSEL stem cells in PRP following 3 minutes of white light treatment is increased to 1.504×10⁶/mL, on Day 1 of culture it is 2.66×10⁵/mL and on day 7 of culture it is 2.18×10⁵/mL. The number of hVSEL stem cells in PRP following no treatment (as a control) is 1.072×10⁶/mL, on Day 1 of culture it is 4.7×10⁵/mL and on day 7 of culture it is 1.657×10⁵/mL.

This embodiment confirms the presence of hVSEL stem cells in whole peripheral blood after red cell lysis. The data shows an increase in hVSEL stem cell numbers in PRP which confirms that PRP is an efficient route to isolate hVSEL stem cells for experimental and clinical use.

The highest numbers of hVSEL stem cells in PRP are found in the Costa laser with SONG modulation with a 3-minute exposure time. The same laser exposure without SONG modulation show fewer hVSEL stem cells but still increased levels over controls indicating some possible benefits of laser exposure even without SONG modulation. The white light and no treatment controls both show fewer hVSEL stem cells than the SONG modulated and SONG unmodulated treatments.

The numbers of hVSEL stem cells present after 1 and 7 days of culture in vitro decreased which may reflect cell death related to in vitro culture.

Time Titration of SONG modulated Magna Costa and Costa Laser on hVSEL Stem Cells in PRP

In an embodiment, the PRP (prepared in accordance with the method of FIG. 3) is exposed to the Magna Costa laser for 3 minutes and the Costa laser for 3, 6 and 9 minutes. White light and no treatment controls are used. hVSEL stem cell flow cytometer analysis is thereafter carried out for all exposure times.

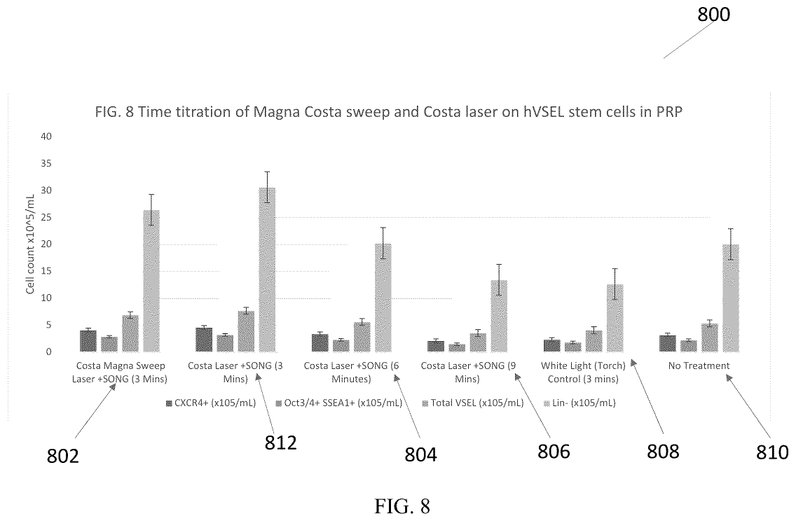

This embodiment is directed towards identifying the optimum laser exposure time for the proliferation of hVSEL stem cells in PRP. FIG. 8 shows a graph 800 illustrating data pertaining to time titration of SONG modulated Magna Costa and Costa Laser on hVSEL Stem Cells in PRP. In embodiments, the laser exposure time ranges from 1 minute to 6 minutes. In embodiments, the laser exposure time ranges from 1 minute to 3 minutes. In embodiments, for a volume ranging from 20 milliliters to 30 milliliters, the laser exposure time is 3 minutes. In other embodiments, laser exposure time is dependent on the volume of PRP. In other embodiments, laser exposure time is dependent on the quality of harvested PRP. As shown, the total hVSEL stem cells found in PRP exposed to the SONG modulated Costa Magna and the Costa laser for three minutes 802 are higher than exposure to the SONG modulated Costa laser for 6 minutes 804 or 9 minutes 806. These data confirm that the optimum laser exposure time to maximize hVSEL stem cell proliferation is 3 minutes. The white light (torch) control 808 and the no treatment control 810 showed hVSEL stem cell numbers less than the 3-minute SONG modulated laser 812 exposure confirming the optimized exposure time to 3 minutes.

Data resulting from various embodiments of the present specification confirm that laser treatment, exposure or modulation of hVSEL stem cells in PRP results in hVSEL stem cell proliferation. This has a great potential in future routine therapy and also in understanding the true nature of hVSEL stem cells.

In embodiments, optimization of a PRP preparation for laser activation of hVSEL stem cells is dependent upon many factors, including, but not limited to centrifugation time, cell collection, the time between laser treatment and patient administration. In addition, in embodiments, a triple shake method may be employed which may a) result in an increase in the yield of the cells which are concentrated at the interface between the plasma and the gel that effectuates the separation, as fewer cells are lost by virtue of being stuck to the interface and b) an increase in cytokines and growth factors that are present in the preparation either before or after laser treatment.

### Unblocking CXCR4 To Make it Available for Binding

Endogenous Peptide Inhibitor X4 (EPI-X4) is the antagonistic ligand of CXCR4. This naturally occurring peptide, originating from the fragmentation of albumin, binds to the CXCR4 antigen mostly by interacting in the minor pocket of CXCR4 through its N-terminal residues, thereby inhibiting G-protein signaling to the associated cells. There have been several EPI-X4 derivatives reported and their IC50 values show that the N-terminal residues of EPI-X4 are crucial for binding to CXCR4.

It has subsequently been shown that the NTer-IN configuration (N-Terminal of EPI-X4 IN the minor pocket of CXCR4) plays a vital role in CXCR4/EPI-X4 binding. Furthermore, only seven EPI-X4 residues played any significant role in this binding, four of which, all positively charged, interact through the minor pocket of CXCR4.

In addition, the negatively charged EPI-X4 residue L16 (C-terminal Leu) interacting with the CXCR4 residue K271 (Lys) has a de-stabilizing effect. However, chemical elimination of L16 showed little effect on the binding of EPI-X4 to CXCR4, demonstrating that first three salt bridges and hydrogen bond are the major agents of the binding.

The last two of the seven significant interactions, V11 and T15 of EPI-X4 interact with E25 and R30, which comprise the β-strand of CXCR4, also providing some small additional binding stabilization. The chemical elimination of EPI-X4 residue L1 or K7 almost completely abolishes receptor binding.

Salt bridges are interactions, electrostatic combined with hydrogen bonding, between oppositely charged residues. Whereas hydrogen bonds can combine, as in water, to create a major force, individual bonds are weak and easily broken. The distance between the residues participating in a salt bridge is important and is typically on the order of <400 picometers (pm). Amino acids greater than this distance apart do not qualify as forming a salt bridge and salt bridges experience thermal fluctuations which continuously break and reform the hydrogen bonds.

EPI-X4, originating from albumin fragmented in the acidic conditions of embryonic gastrulation, binds to and dysregulates the CXCR4 expressed by the hVSEL stem cells, protecting the salt bridges and hydrogen bonds in the minor pocket of CXCR4 from thermal fluctuations, thereby maintaining hVSEL stem cell quiescence.

CXCR4 is unblocked by SONG modulated laser light to make it readily available for binding by flow cytometry antibodies. The SONG modulated red laser penetrates the minor pocket of CXCR4 and thus disrupts the hydrogen bonds and salt bridges binding CXCR4 to EPI-X4. A three-minute exposure time to SONG modulated laser is observed to be most effective in unblocking CXCR4. In the given time, the laser thermal turbulence in the minor pocket of CXCR4 maximizes the proliferation of hVSEL stem cells in vitro. In three minutes, the binding of EPI-X4 to CXCR4 is broken and the laser becomes ineffective because the thermal energy of the minor pocket is comparable to that of the red-energy laser.

After three minutes of continuous laser exposure, a new thermal stability is established as the turbulent conditions subside and new hydrogen bonds, if not salt bridges, develop in the hotter but now-stabilizing conditions. When the laser is applied for six and nine minutes, the hVSEL count decreases as the hotter stabilizing conditions in the minor pocket of CXCR4 allow some new hydrogen bonds, which demonstrably show some re-binding effect across the CXCR4/EPI-X4 complex.

The apparent rapid proliferation of hVSEL stem cells in PRP in vitro demonstrates that the SONG modulated red laser for three minutes penetrates into the minor pocket of CXCR4 and interrupts the salt bridges and the hydrogen bonds, thus breaking the CXCR4/EPI-X4 binding and exposing CXCR4 to labelled antibodies in the subsequent flow cytometry analysis.

### Methods of Using Modulated Laser Photobiomodulation

The present specification describes a method of using modulated laser photobiomodulation to increase the proliferation of peripheral blood hVSEL stem cells, harvesting and storing the hVSEL for subsequent use. As described above, embryonic-like (hVSEL) stem cells are produced by migrating PGCs, and have been shown to be capable of producing hemopoietic stem cells (HSC) and endothelial progenitor cells (EPC). It has been established that hVSEL stem cells, since they are pluripotent, may be the precursors of other stem cells in the body. hVSEL stem cells also persist in peripheral blood throughout life. Hence, it is possible to obtain autologous hVSEL stem cells from any patient at any age.

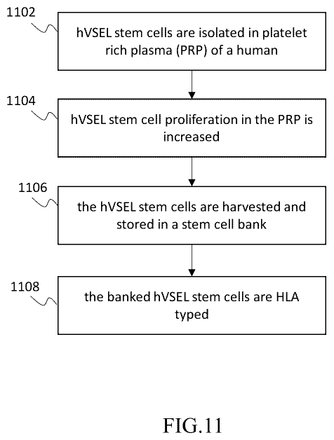

FIG. 11 illustrates a method of harvesting and storing hVSEL stem cells for subsequent use, in accordance with an embodiment of the present specification. At step 1102 hVSEL stem cells are isolated in the platelet rich plasma (PRP) of a human. As described earlier in the specification, there is a constant availability of hVSEL stem cells in the collected PRP of patients who have undergone multiple PRP collections. PRP containing hVSEL stem cells is prepared to facilitate collection of the isolated hVSEL stem cells. At step 1104, hVSEL stem cell proliferation in the PRP is increased. In accordance with aspects of the present specification, in order to increase stem cell proliferation, PRP containing hVSEL stem cells is manipulated or modified using pulses of laser light having a wavelength in a range of 300 nm to 1000 nm, and, in an embodiment, approximately 670 nm. Embodiments of the present specification use SONG modulated laser light to interact with hVSEL in PRP to upregulate the expression of CXCR4, Oct 3/4 and SSEA4, as described with reference to FIG. 3 above. In some embodiments, to assess the biological stability of the effect of laser exposure or manipulation, the PRP is cultured in equal volumes of RPMI 1640 media supplemented with 200 mM L-Glutamine, penicillin, and streptomycin and 10% heat inactivated fetal calf serum.

At step 1106 the hVSEL stem cells are harvested and stored in a stem cell bank, as described above. As described above, the laser has a proliferative effect on hVSEL stem cells in PRP. This effect is maintained in relative terms for at least 24 hours in vitro post laser exposure. In embodiments, stem cell administration may occur in a time frame wherein the measurable effect on hVSEL remains post laser exposure, wherein said time may vary depending on a plurality of conditions. At step 1108, the banked hVSEL stem cells undergo HLA typing, which, in this embodiment, occurs after laser exposure but may also occur before laser exposure. As is known, Human leukocyte antigen (HLA) typing is used to match patients and donors for bone marrow or cord blood transplants. HLA are proteins, or markers, found on most cells in a human body form the major histocompatibility complex. In embodiments, the harvested and stored hVSEL cells are HLA typed to facilitate organ transplant by using said cells.

### Use Case 1: Intrinsic Age Reduction

The method of the present specification provides regenerative medicine using allogeneic hVSEL stem cells that are younger than the patient's own hVSEL stem cells. As described above autologous hVSEL stem cells may be obtained from any patient at any age, thereby enabling their use in regenerative medicine, simplifying procedures, saving money and reducing adverse reactions associated with allogeneic cells. While previously believed to be biologically ageless, recent research indicates that hVSEL stem cells may age, but at a much slower rate than the corresponding individual. For example, allogeneic hVSEL stem cells of a patient aged 80 years may be aged only 50 years, and hence may be used to effectively drive the patient's biological age lower than would be otherwise possible. Allogenic hVSEL may be obtained from a younger donor, such as, but not limited to, from umbilical chord of said donor. In an embodiment, a youngest hVSEL stem cell match for a patient may be obtained from the hVSEL stem cell bank as described above. The match may be obtained by using known HLA-typing techniques and by using flow cytometry to assess the characteristic surface protein patterns of the matched hVSEL stem cells. As described above, flow cytometry is often used to assess laser treated biological samples for cell proliferation. Surface antigens Oct 3/4, SSEA4 and CXCR4 in the lineage negative (Lin−) compartment are assessed using flow cytometry. In embodiments, each sample of hVSEL stem cells obtained from the stem cell bank is assessed for hVSEL stem cell numbers using the flow cytometry protocol mentioned earlier in this specification.

Intrinsic epigenetic age (IEA) is a true indicator of biological age at the DNA level. In one embodiment, by using the treatment and administration procedures described herein, in one embodiment, a single treatment, as described herein, can yield a reduction in an individual's IEA by 2 to 4 years, a second treatment can yield an additional reduction in an individual's IEA by 2 to 4 years, a third treatment can yield an additional reduction in an individual's LEA by 2 to 4 years, and a fourth treatment can yield an additional reduction in an individual's IEA by 2 to 4 years. Accordingly, for each treatment, the IEA may reduce by 2 years to 4 years such that four treatments, spread over a period of 1 month to 24 months can yield a reduction in an individual's IEA by 8 to 16 years. By way of another example, in using the treatment and administration procedures as described herein, an individual's IEA may be decreased in a range of 1 year to 4 years. More specifically, in one embodiment, a single treatment, as described herein, can yield a reduction in an individual's IEA by 1 to 4 years, a second treatment can yield an additional reduction in an individual's IEA by 1 to 5 years, a third treatment can yield an additional reduction in an individual's IEA by 1 to 5 years, and a fourth treatment can yield an additional reduction in an individual's IEA by 1 to 5 years. Accordingly, for each treatment, the IEA may reduce by 1 year to 5 years such that four treatments, spread over a period of 1 month to 24 months can yield a reduction in an individual's IEA by 4 to 20 years. In embodiments, treatments are administered every week to every year and in any increment therein. In embodiments, treatments are administered every week to every six months and in any increment therein. Optionally, treatments may be administered in any frequency as long as it achieves the objectives of the present specification.

In embodiments, a patient experiences a decrease in biological age in a range of 1 year to 4 years based on a first administration of the treated platelet rich plasma. In embodiments, a patient experiences a decrease in biological age in a range of 4 years to 9 years based on a second administration of the treated platelet rich plasma. In embodiments, the second administration of the treated platelet rich plasma occurs 1 week to 6 months after the first administration.

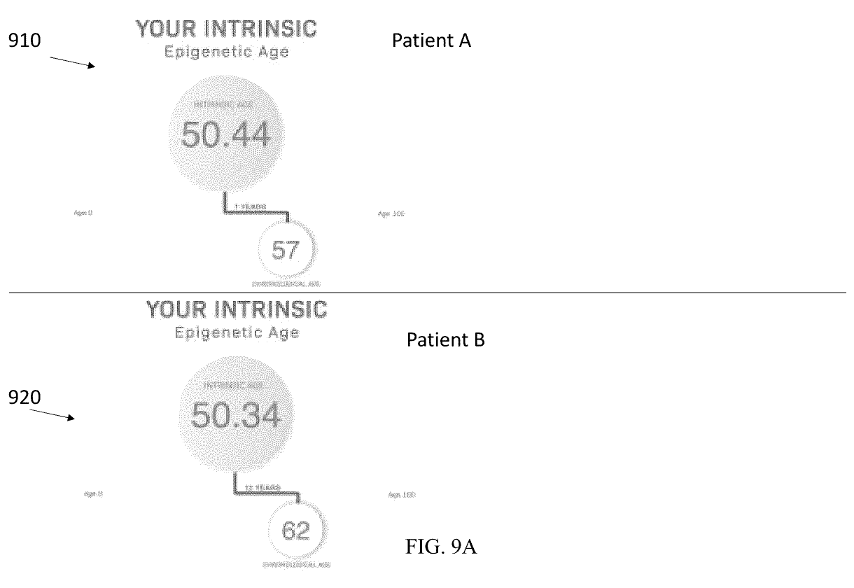

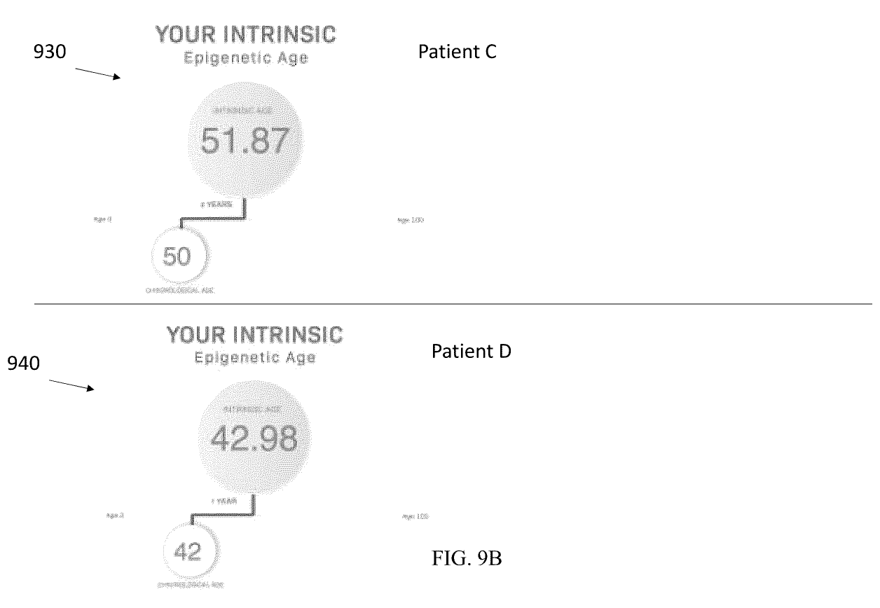

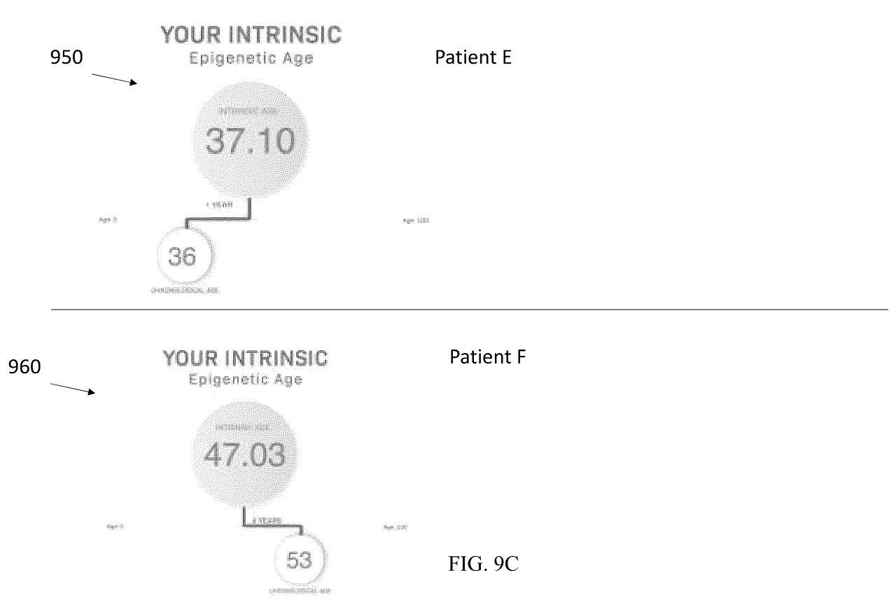

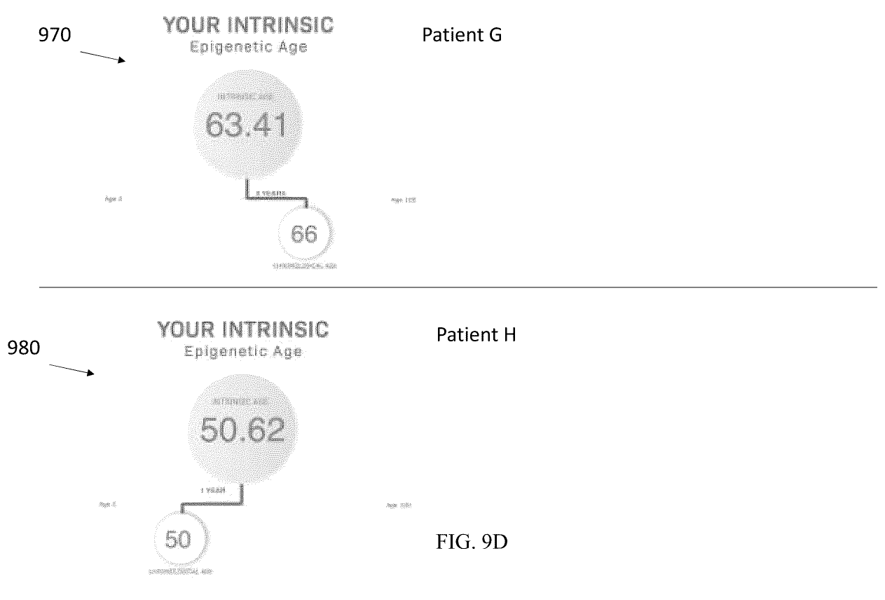

Referring to FIGS. 9A to 9D, examples of intrinsic epigenetic ages determined for eight different humans, referred to as patient A through patient H, are provided. FIG. 9A shows an IEA of patient A 910 at 50.44 years when patient A's chronological age is 57 years. The figure also illustrates an IEA of patient B 920 at 50.34 years, when patient B's chronological age is 62 years. FIG. 9B shows an IEA of patient C 930 at 51.87 years, when patient C's chronological age is 50 years. The figure also illustrates an IEA of patient D 940 at 42.98 years, when patient D's chronological age is 42 years. FIG. 9C shows an IEA of patient E 950 at 37.10 years, when patient E+s chronological age is 36 years. The figure also illustrates IEA of patient F 960 at 47.03 years, when patient F's chronological age is 53 years. FIG. 9D shows an IEA of patient G 970 at 63.41 years, when patient G's chronological age is 66 years. The figure also illustrates an IEA of patient H 980 at 50.62 years, when patient H's chronological age is 50 years. Therefore, chronological age can be very different from the biological age, which can further be different for IEA and EEA.

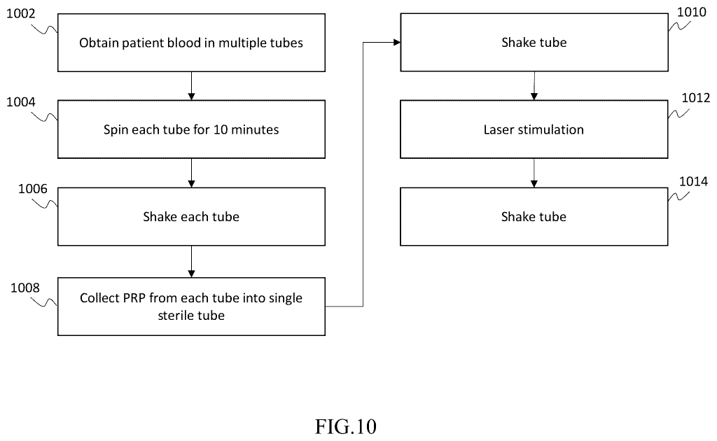

It is desirable to reduce the speed of, inhibit, or even reverse epigenetic aging in general, and IE aging, in particular. To do so, a treatment as shown in FIG. 10 is administered to each patient. FIG. 10 is a flow chart illustrating an exemplary process of preparing PRP that contains hVSEL stem cells, which are used for reducing IEA, in accordance with some embodiments of the present specification. IEA is reduced by increasing regenerative growth factors resulting from proliferation of hVSEL stem cells. At step 1002, a patient's blood is obtained in a plurality of tubes of 10 cc each. In embodiments, the number of tubes ranges from 3 to 12. In a preferred embodiment, a patient's blood is obtained in six tubes of 10 cc each. At step 1004, each tube is spun with a centrifugal G-force of approximately 270G for approximately 10 minutes. The spinning process pulls red/white blood cells into a gel at the bottom of each tube. Platelets, hVSEL stem cells, and plasma remain separated above the gel in the form of PRP. Based on basic density distribution, an upper third of the PRP has the lowest concentration of hVSEL stem cells, while a bottom third of PRP, near the gel interface, has the highest concentration of hVSEL stem cells. At step 1006, the tubes are shaken for a first time. The shaking involves gently rocking the tube in a back-and-forth motion for approximately 10 seconds. The motion knocks loose hVSEL stem cells in PRP near the gel boundary, thereby improving hVSEL yield by at least 1% compared to an identical procedure where no such shaking is performed. At step 1008, approximately 6 to 7 cc of PRP per tube is harvested. The harvested amounts are collected in a separate sterile tube. At step 1010, the tube containing the PRP harvested at step 1008 is shaken (second shaking). In some embodiments, the shaking is performed vigorously for approximately 20-50 seconds, and preferably 30 seconds, to release regenerative factors. At step 1012, laser stimulation is applied. Laser stimulation is applied as described above. In some embodiments, SONG modulated laser stimulation is applied for three minutes. At step 1014, the tube is shaken for a third time for approximately 20-50 seconds, and preferably 30 seconds to awaken dormant hVSEL stem cells and generate cytokines and growth factors from hVSEL stem cells.

FIG. 10 provides an exemplary process of treating PRP obtained from blood samples of a patient. Variations to the process without deviating from the scope of the present invention are also possible to reduce IEA. Reduction in biological age may increase on a per treatment basis.

The treatment described in FIG. 10 is repeated to achieve additional reductions in the biological age. Therefore, in an example, one treatment may decrease biological age by one year while an additional treatment may decrease it by an additional year. The amount of reduction in IEA achieved by embodiments of the present specification is higher than any other known treatment.

Accordingly, referring back to the case examples provided in FIGS. 9A to 9D, if patient A is provided one treatment, the intrinsic epigenic age reduces from 50.44 years to approximately 48 to 46 years old. If patient B is provided two treatments, spread apart by a time period ranging from 1 week to 6 months, the chronological age reduces from 50.34 years to approximately 46 to 42 years old. If patient C is provided three treatments, each spread apart by a time period ranging from 1 week to 6 months, the chronological age reduces from 51.87 years to approximately 46 to 40 years old. If patient D is provided four treatments, each spread apart by a time period ranging from 1 week to 6 months, the chronological age reduces from 52.98 years to approximately 45 to 37 years old. If patient E is provided five treatments, each spread apart by a time period ranging from 1 week to 6 months, the chronological age reduces from 37.10 years to approximately 27 to 17 years old. If patient F is provided six treatments, each spread apart by a time period ranging from 1 week to 6 months, the chronological age reduces from 47.03 years to approximately 35 to 23 years old. Finally, if patient G is provided seven treatments, each spread apart by a time period ranging from 1 week to 6 months, the chronological age reduces from 63.41 years to approximately 49 to 35 years old.

Use Case 2: Treatment of Cardiac Disease and/or End Stage Heart Failure

The use of multiple stem cell types (for example, bone marrow MSC and CD34+ hemopoietic stem cells (HSC) together) may be effective in the repair of the deficient stem cell niche and reduced stem cell numbers often seen in disease. This is because both MSC and CD34+ HSC are multipotent and, when used in combination, the overall efficacy, is increased. Preparations of both of these cell types (MSC and HSC) also contain increased numbers of hVSEL stem cells, which may contribute to the apparent increase in efficacy.

The hVSEL stem cells are pluripotent and therefore have considerable potential in the regeneration of damaged organs after exposed to SONG modulated laser light. The small size (1-4 μm in diameter) of hVSEL stem cells gives them the added advantage that they can cross the blood brain barrier and do not become trapped in the various capillary beds when administered intravenously.

In embodiments, allogeneic NPT is used to treat cord blood derived MSC in the treatment of end-stage heart failure. Further NPT is used to treat autologous hVSEL stem cells in the treatment of end-stage heart failure. In a study, patients treated for end-stage heart failure using autologous hVSEL stem cells also showed significant enhancement of LV function, including normalization of and stabilization of LVEF following treatment.

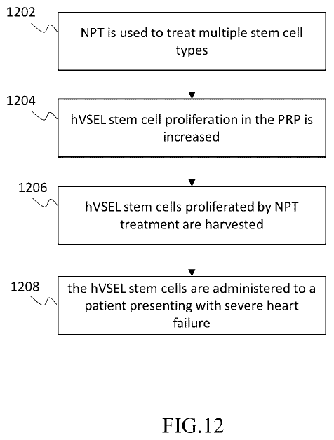

FIG. 12 is a flowchart illustrating a method of treating end stage heart failure, in accordance with an embodiment of the present specification. At step 1202, NPT is used to treat multiple stem cell types. At step 1204, hVSEL stem cell proliferation in the PRP is increased using a form of NPT, and specifically, the use of a SONG modulated laser to activate hVSEL stem cells in PRP, including SONG modulated laser being applied to stem cells in vitro and directly to the patient, as described above. In embodiments, NPT is used to treat stem cell types such as, but not limited to bone marrow MSC and CD34+ hemopoietic stem cells (HSC) together. In an embodiment, allogeneic NPT is used to treat cord blood derived MSC. At step 1206, hVSEL stem cells proliferated by NPT treatment are harvested. At step 1208 the hVSEL stem cells are administered to a patient presenting with severe heart failure.

In one study, SONG modulated laser treated autologous hVSEL stem cells were used to treat heart failure in a 61 year-old male with a LVEF of 18% awaiting a heart transplant. The prognosis was 30% survival at one year without treatment. The first treatment for this patient used SONG modulated laser treated allogeneic expanded cord blood MSC. Subsequently, he received annual treatment using SONG modulated laser treated autologous hVSEL stem cells and his LVEF has maintained within a range of 38-40%.

In another case, a patient presented with severe heart failure with a LVEF ranging between He was 66 years old at the time of his SONG modulated laser treated autologous hVSEL procedure in April 2017. In late 2017, he was able to stop taking all of his cardiovascular and anti-hypertensive medications including Ramipril, Carvedilol, low dose Aspirin and Clopidogrel. In December 2013, (prior to the autologous hVSEL stem cell procedure) he had an Implantable Cardioverter-Defibrillator (ICD) inserted, which has remained in place but will be removed when the battery charge runs out at his next cardiologist visit. He most recent LVEF was 37%, without any medications.

The SONG modulated laser treated hVSEL stem cells are also capable of repairing the components of the stem cell niche thus re-enabling the long-term survival of existing endogenous stem cells and new cardiac stem cells derived from exogenous hVSEL stem cells. PRP is a concentrated and complex mixture of cytokines and growth factors which has the potential to enhance the process of stem cell niche repair. PRP contains large numbers of platelets which produce various biologically active molecules through the secretion of alpha granules. Induced pluripotent stem cell derived cardiomyocytes are known to be capable of secreting exosomes which contribute towards myocardial repair. Therefore, exosomes, produced by hVSEL stem cells, contribute to the regenerative effect seen.

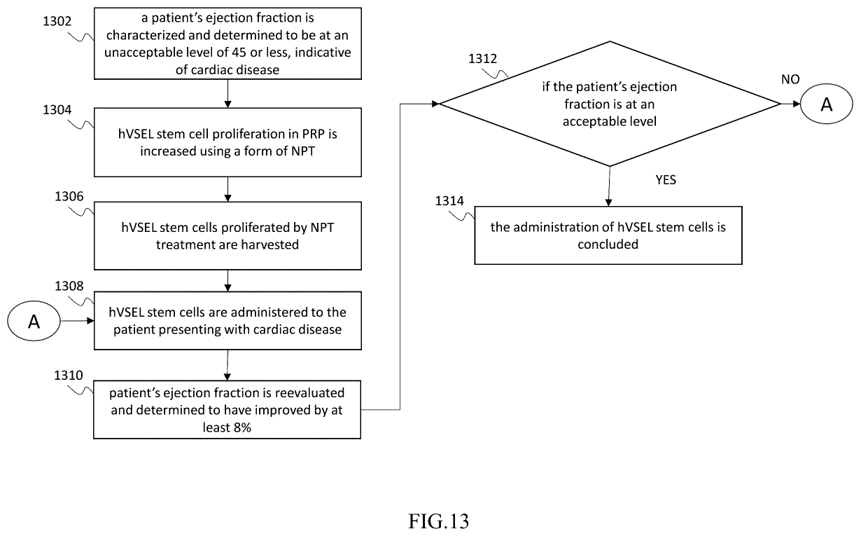

FIG. 13 is a flowchart illustrating a method of treating cardiac disease whereby a disease state is characterized by a low ejection fraction, in accordance with an embodiment of the present specification. At step 1302, in an embodiment, a patient's ejection fraction is characterized and determined to be at a level of 45 or less, which is low and unacceptable, indicative of cardiac disease. At step 1304, hVSEL stem cell proliferation in PRP is increased using a form of NPT, as described above. In embodiments, a SONG modulated laser is used to activate hVSEL stem cells in PRP. In an embodiment, the SONG modulated laser is applied to stem cells in vitro and/or directly to the patient, as described above. In embodiments, NPT is used to treat stem cell types such as, but not limited to bone marrow MSC and CD34+ hemopoietic stem cells (HSC) together. In an embodiment, NPT is used to treat cord blood derived MSC. At step 1306, hVSEL stem cells proliferated by NPT treatment are harvested. At step 1308, the hVSEL stem cells are administered to the patient presenting with cardiac disease. At step 1310, the patient's ejection fraction is reevaluated and determined to have improved by at least 8%, and preferably by at least 10%. At step 1312, it is determined if the patient's ejection fraction is at an acceptable level. If the patient's ejection fraction is not at an acceptable level, then steps 1308 to 1312 are repeated at predefined intervals of time. If the patient's ejection fraction is at an acceptable level, the treatment is stopped at step 1314. In an embodiment, after every administration of hVSEL stem cells, the patient's ejection fraction improves by a minimum ranging from 8% to 10%. In an embodiment, steps 1308 to 1312 are repeated after an elapsed time period in a range of 1 day, 1 week, 1 month, 3 months, 6 months, or any time increment or range therein for the purpose of increasing the patient's ejection fraction to an acceptable level. Hence, in embodiments, the procedure of administrating hVSEL stem cells is continuously repeated, with each subsequent treatment occurring after the elapsed period, to achieve a step-wise improvement in ejection fraction of at least 8% at each step until the desired ejection fraction which is greater than 45 is achieved.

It should be appreciated that neurodegenerative disease, neurological trauma, type 2 diabetes, liver disease, lung disease, pancreatic disease, and psychosis and psychiatric disorders may be similarly treated. First, the disease state is characterized by the measurement of a biomarker (i.e. insulin level, hemoglobin A1C level, oxidative stress markers, serum biomarkers such as alanine aminotransferase (ALT), aspartate aminotransferase (AST), alkaline phosphatase (ALP), glutamyl transpeptidase (GGT), and total bilirubin (TBIL), antigens, such as carbohydrate antigen (CA)) and/or characterization of tissue. If the biomarker or tissue has a level or type that is indicative of one of the aforementioned diseases, the patient's platelet rich plasma is obtained and hVSEL stem cell proliferation in PRP is increased using a form of NPT, as described above. In embodiments, a SONG modulated laser is used to activate hVSEL stem cells in PRP. In an embodiment, the SONG modulated laser is applied to stem cells in vitro and/or directly to the patient, as described above. In embodiments, NPT is used to treat stem cell types such as, but not limited to bone marrow MSC and CD34+ hemopoietic stem cells (HSC) together. In an embodiment, NPT is used to treat cord blood derived MSC. The hVSEL stem cells proliferated by NPT treatment are harvested. The hVSEL stem cells are administered to the patient presenting with said disease. The patient's biomarkers and/or tissue types are reevaluated and determined to have improved by at least 8%, and preferably by at least 10%. If the patient's biomarker or tissue type is not at an acceptable level or state, then the above steps are repeated at predefined intervals of time. If the patient's biomarker or tissue type is at an acceptable level, the treatment is stopped. In an embodiment, after every administration of hVSEL stem cells, the patient's biomarker level or tissue type improves by a minimum ranging from 8% to 10%. In an embodiment, the above steps are repeated after an elapsed time period in a range of 1 day, 1 week, 1 month, 3 months, 6 months, or any time increment or range therein for the purpose of improving the patient's biomarker level to an acceptable level. Hence, in embodiments, the procedure of administrating hVSEL stem cells is continuously repeated, with each subsequent treatment occurring after the elapsed period, to achieve a step-wise improvement of at least 8% at each step until the desired biomarker level is achieved.

It should further be appreciated that the present application is generally directed toward determining a quantitative measure of a disease state, such as an amount of tissue, a biomarker, or other composition, determining the patient is in a disease state, obtaining autologous VSELs, applying the NPT process as described above, harvesting the proliferated and activated stem cells, administering the proliferated and activated stem cells to the patient, reevaluating the quantitative measure again to determine if at least 8% improvement is achieved, and repeating the process until the quantitative measure reaches an acceptable level.

As described throughout the specification, both disease and aging can be correlated to a reduction in endogenous stem cells and a degradation of the stem cell niche. The number of stem cells available has also been shown to decline in pediatric heart disease along with the suggestion that autologous supplementation of stem cells may be beneficial. In the patient suffering from heart failure, it may be assumed that as part of the disease process, endogenous stem cell numbers are decreased and the stem cell niche has been degraded or damaged. Direct regeneration of cardiac somatic cells by exogenous stem cells of hVSEL, where the underlying problems are reduced stem cell numbers and a damaged stem cell niche, is likely to have a transient benefit to the patient. hVSEL stem cells are pluripotent and can produce any tissue including cardiomyocytes. Additionally, hVSEL stem cells directly replenish the decreased endogenous stem cell reserve in disease thus facilitating a long-term benefit to patients.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method of treatment of a disease of a patient, wherein said disease is characterized by at least one unique marker, comprising:
determining the patient has said disease based at least in part on a value of said at least one unique marker;
proliferating stem cells of the patient, wherein said proliferation comprises:
obtaining platelet rich plasma from the patient, wherein the platelet rich plasma has a first quantity of stem cells;
exposing the platelet rich plasma with the first quantity of stem cells to modulated pulses of laser light having a predefined wavelength and for a predefined period of time; and,
after said exposure, harvesting from said platelet rich plasma a second quantity of stem cells, wherein the second quantity of stem cells is greater than the first quantity;
administering a composition comprising, at least in part, said second quantity of stem cells;
evaluating said at least one unique marker;
determining whether said at least one unique marker is at an acceptable level;
if said at least one unique marker is at an acceptable level, ending said method of treatment; and
if said at least one unique marker is not at an acceptable level, waiting a period of time and repeating each of the determining, proliferating and administering steps.

2. The method of claim 1, wherein preparing the platelet rich plasma comprises:
placing the patient's blood into a plurality of tubes;
centrifuging the plurality of tubes for a predefined period of time to produce the platelet rich plasma; and
aliquoting the produced platelet rich plasma into a sterile tube.

3. The method of claim 2, further comprising shaking the sterile tube after aliquoting.

4. The method of claim 1, further comprising shaking the platelet rich plasma after treating with modulated pulses of laser light.

5. The method of claim 1, wherein the predefined wavelength ranges from 300 nm to 1000 nm.

6. The method of claim 1, wherein the predefined wavelength is in a range of 580 nm to 770 nm.

7. The method of claim 1, wherein the predefined period of time ranges from 1 minute to 5 minutes.

8. The method of claim 1, wherein the second quantity of stem cells is greater than the first quantity by 10% up to 400%.

9. The method of claim 1, wherein said repeating each of the determining, proliferating and administering steps occurs 1 day to 6 months after said administering of the composition comprising, at least in part, said second quantity of stem cells.

10. The method of claim 1, wherein the marker is an ejection fraction percentage and wherein said ejection fraction percentage increases by at least 8% after said administering of the composition comprising, at least in part, said second quantity of stem cells.

11. The method of claim 1, wherein the marker is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and wherein said marker improves by at least 8% after said administering of the composition comprising, at least in part, said second quantity of stem cells.

12. The method of claim 1, wherein the disease comprises at least one of a cardiac disease, a neurodegenerative disease, a musculoskeletal trauma, a neurological trauma, type 2 diabetes, liver disease, lung disease, pancreatic disease, a psychosis disorder, or a psychiatric disorder.

13. A method of treating a disease in a patient characterized by a predefined parameter, the method comprising:
characterizing the predefined parameter for determining said parameter to be out of a predefined acceptable parameter range;
obtaining platelet rich plasma comprising a first quantity of autologous hVSELs from the patient;
increasing a number of said hVSEL stem cells from the first quantity to a second quantity by applying to said platelet rich plasma pulses of modulated laser light;
harvesting the second quantity of hVSEL stem cells;
administering the second quantity of hVSEL stem cells to the patient;
reevaluating the patient's predefined parameter;
determining if the patient's predefined parameter is at an acceptable level;
repeating at predefined intervals of time each of the obtaining, increasing, harvesting, and administering steps if the patient's predefined parameter is not at the acceptable level; and
concluding said method of treating the patient if the patient's predefined parameter is at the acceptable level.

14. The method of claim 13, wherein after performing each of the obtaining, increasing, harvesting, and administering steps, the patient's predefined parameter improves by at least 8%. The method of claim 13, wherein the second quantity of hVSEL stem cells is greater than the first quantity by 10% up to 400%.

16. The method of claim 13, wherein said predefined intervals of time are in a range of 1 day to 6 months, or any time increment therein.

17. The method of claim 13, wherein the pulses of modulated laser light have a predefined wavelength in a range of 580 nm to 770 nm.

18. The method of claim 13, wherein the predefined parameter is an ejection fraction percentage and wherein said ejection fraction percentage increases by at least 8% after said administering the second quantity of hVSEL stem cells.

19. The method of claim 13, wherein the predefined parameter is at least one of an insulin level, a hemoglobin A1C level, an oxidative stress marker level, a serum biomarker level, an alanine aminotransferase level, an aspartate aminotransferase level, an alkaline phosphatase level, a glutamyl transpeptidase level, a total bilirubin level, or an antigen level and wherein said predefined parameter improves by at least 8% after said administering the second quantity of hVSEL stem cells. The method of claim 13, wherein the disease comprises at least one of a cardiac disease, a neurodegenerative disease, a musculoskeletal trauma, a neurological trauma, type 2 diabetes, liver disease, lung disease, pancreatic disease, a psychosis disorder or a psychiatric disorder.
